(19) United States
(12) Patent Application Publication
(10) Pub. No.: US 2015/0268223 A1
(43) Pub. Date: Sep. 24, 2015

(54) MICROPATTERNED CO-CULTURE SYSTEMS AS INFECTIOUS DISEASE ANALYSIS PLATFORMS

(71) Applicant: Massachusetts Institute of Technology, Cambridge, MA (US)

(72) Inventors: Sangeeta N. BHATIA, Lexington, MA (US); Salman R. KHETANI, Fort Collins, CO (US)

(21) Appl. No.: 14/573,731

(22) Filed: Dec. 17, 2014

Related U.S. Application Data
(62) Division of application No. 13/003,214, filed on Jul. 5, 2011.
(60) Provisional application No. 61/174,449, filed on Apr. 30, 2009.
(60) Provisional application No. 61/078,683, filed on Jul. 7, 2008.

(51) Int. Cl. G01N 33/50; C12Q 1/18

(52) U.S. Cl. CPC G01N 33/5067; G01N 33/5014; C12Q 1/18

(57) ABSTRACT

Cell cultures are provided that include a population of micropatterened hepatocytes and one or more non-parenchymal cell populations, where the hepatocytes are infected with a virus or parasite and include a reporter of virus or parasite infection. Methods of making and using the cell cultures are also provided.

### RELATED APPLICATIONS

This application is a divisional of U.S. Utility application Ser. No. 13/003,214, filed Jul. 5, 2011, which claims priority to International Application No. PCT/US2009/049849, filed Jul. 7, 2009, which claims priority to U.S. provisional application Ser. No. 61/078,683 filed Jul. 7, 2008 and U.S. provisional application Ser. No. 61/174,449 filed Apr. 30, 2009. The entire contents of each of the foregoing applications are incorporated herein by this reference.

### BACKGROUND

More than 150 million people worldwide are infected with the Hepatitis C virus (HCV), and about 75% of those infected display chronic infection, which can lead to liver cirrhosis and hepatocarcinoma. Current drug therapies (pegylated interferon and ribavirin) have severe side effects and do not cure many infected patients even after lengthy (months to year) treatment regimens. Hence, there is an urgent need to better understand the variability in host response and propensity for chronic HCV infection, and then use such understanding to develop new classes of drugs that target specific pathways in the viral lifecycle. However, these goals have been difficult to realize partly due to the difficulties in maintaining sustained and reproducible infection of isolated primary human hepatocytes (main cell type of the liver) with HCV in vitro. Recently, a human hepatoma cell line (Huh-7.5) was shown to be susceptible to HCV virus and produce viral particles de novo. However, this cancer-derived cell line has abnormal repertoire and levels of liver-specific functions. Hence, primary human hepatocytes are considered ideal for faithfully mimicking physiologically-relevant host responses to HCV.

Similar difficulties exist for the study of other infectious diseases which infect the liver, e.g., members of the Flavivirus genus, Plasmodium falciparum, Plasmodium vivax, Plasmodium ovale, Plasmodium malariae, and hepatitis A, B, δ, D, E. As an example, great efforts are currently being made to develop vaccines for malaria. Often the vaccine is an attenuated virus, and it is critical to insure that the vaccine arrests the development of the malaria parasite at the liver stage (i.e., before it breaks free of liver hepatocytes to infect red blood cells). Monitoring molecular biology of the parasite in the liver is difficult using current methods, and there is no easy way to determine in cell culture whether the vaccine is properly arresting the parasite.

Historically cell culture techniques and tissue development failed to take into account the necessary microenvironment for cell-cell and cell-matrix communication as well as an adequate diffusional environment for delivery of nutrients and removal of waste products. Cell culture techniques and understanding of the complex interactions cells have with one another and the surrounding environment have improved in the past decade.

While many methods and bioreactors have been developed to grow tissue for the purposes of generating artificial tissues for transplantation or for toxicology studies, these bioreactors do not adequately simulate, in vitro, the mechanisms by which nutrients, gases, and cell-cell interactions are delivered and performed in vivo. For example, cells in living tissue are “polarized” with respect to diffusion gradients. Differential delivery of oxygen and nutrients, as occurs in vivo by means of the capillary system, controls the relative functions of tissue cells and their maturation. Thus, cell culture systems and bioreactors that do not simulate these in vivo delivery mechanisms do not provide a sufficient corollary to in vivo environments to develop tissues or measure tissue responses in vitro. Further, traditional cell culture systems often fail to provide adequate information on the liver toxicity and bioavailability of drug candidates. These issues have caused 50% of new drug candidates to fail in Phase I clinical trials. Also, a third of drug withdrawals from the market and more than half of all warning labels on approved drugs are primarily due to adverse affects on the liver.

Current in vitro liver models used by the pharmaceutical industry, though useful in a limited capacity, are not fully predictive of in vivo liver metabolism and toxicity. Thus, research has increasingly turned towards using isolated primary human hepatocytes as the gold standard for in vitro studies; however, hepatocytes are notoriously difficult to maintain in culture as they rapidly lose viability and phenotypic functions.

Recent advances using micropatterned co-cultures of primary human hepatocytes and stromal cells in a multi-well format have overcome many of the limitations of previous techniques, particularly by allowing extended studies of hepatocytes which behave similarly to in vivo liver tissue.

Nevertheless, monitoring of HCV infection and treatment poses specific challenges in cell culture techniques. Firstly, HCV has a low infectivity of cells in culture, making the preparation of in vitro culture systems difficult or impossible. Secondly, monitoring HCV replication is destructive, as the cells are typically destroyed before assessing HCV RNA levels. New methods and protocols are needed to effectively monitor HCV infection, and infection by other pathogenic agents, in cell culture to allow the development and testing of safe and effective therapeutics.

### SUMMARY OF THE INVENTION

The present invention is based, at least in part, on the development and optimization of a disease platform for infectious disease (e.g., HCV) drug development that couples micropatterned co-cultures of primary human hepatocytes and supporting non-parenchymal cells (e.g., stromal cells) in a high-throughput format (e.g., a multi-well format) with highly sensitive viral reporter systems and optimized treatment protocols. The methods, compositions, and micropatterned co-culture systems described herein may be used to investigate diseases of tissues beyond the liver, e.g., by culturing brain cells, pancreatic cells, muscle cells, bone marrow cells, etc., and, in some embodiments, infecting those cultures with infectious agents specific for those tissues.

The invention is further based on non-destructive methodologies (e.g., reporter systems) which may be used in conjunction with the micro-patterned co-cultures, allowing high throughput experimentation and automated optical readout.

### BRIEF DESCRIPTION OF THE DRAWINGS

FIG. 1 depicts microscale human liver tissues (micropatterned co-cultures of primary human hepatocytes with 3T3-J2 murine embryonic fibroblasts) in a 96-well format for higher-throughput screening.

FIG. 2A depicts infection of microscale human liver tissues with retroviral pseudoparticles bearing either the E1/E2 glycoproteins of the Hepatitis C virus (HCV) genotype 1a strain (H77) or the ‘g’ glycoprotein of the vesicular stomatitis virus (VSV), and containing a GFP reporter gene. Cells were infected for 6 hours with the pseudoparticles in the presence of either non-specific IgG antibody or antibody specific to the HCV entry protein, CD81 (Anti-CD81, clone JS-81) at different concentrations (1 ug/mL and 10 ug/mL). 48 hours after infection, the GFP signal indicating infected cells was monitored. The Anti-CD81 antibody blocked entry of H77 HCV pseudo-particles, but not of the VSVg particles suggesting that the entry of HCV-like particles is mediated by CD81 in microscale human liver tissues.

FIG. 2B depicts quantification of images in FIG. 2A. 0, 1 and 10 indicate concentrations of Anti-C81 antibody, while ‘NE’ indicates pseudoparticles that lack glycoproteins and GFP reporter. ‘Mock’ indicates those culture wells that were never incubated with any pseudoparticles (H77, VSVg or NE).

FIG. 3 depicts infection and replication of HCV in microscale human liver tissues and blocking of replication via type I interferon (component of current standard of care therapy) and small molecule inhibitors of proteins implicated in HCV replication (see table above). Microscale tissues were allowed 6 days to stabilize in their liver-specific functions post hepatocyte seeding, and then infected with the J6.Jc1/Flag2/p7GlucNS2 (genotype 2a HCV virus containing luciferase reporter) for 6 hours followed by washing of the cultures. At 24 hours after infection, the culture medium was sampled for assessment of luciferase activity (an indicator of HCV RNA replication). At 48 hours after infection, the culture medium was sampled, cultures were washed 3 times with fresh culture medium, and then the fresh culture medium on the cells was sampled as well to provide ‘zeroing’ of the signal (as indicated by ‘p’ after hours post infection). This procedure was repeated for two weeks.

FIG. 4 shows the effect of dose and time of incubation with initial viral inoculum on the production of HCV particles in the culture medium of microscale human liver tissues. Cultures were infected with HCV as stated in the caption of FIG. 3; however, the initial viral inoculum was a) diluted up to ⅛ the original stock, and b) left on the cultures for 8, 16 or 24 hours, followed by washing of the cultures 3 times with fresh culture medium lacking HCV. Luciferase activity was assessed 72 hours after infection.

FIG. 5A depicts infection of microscale human liver tissues and conventional pure hepatocyte monolayers on collagen-coated tissue culture plastic with HCV. Cultures were infected with HCV as in FIG. 3 and luciferase activity was monitored in culture supernatant over 2 weeks post-infection. The bar graph above shows that microsale human liver tissues produce as much as 8.5 fold more secreted luciferase (a sensitive measure of HCV infection) than conventional monolayers, thereby providing a better signal to noise ratio (noise being mock control values) for testing the effects of potential HCV drugs on production of the virus in hepatocytes.

FIG. 5B depicts the production of infectious HCV by micropatterned primary hepatocyte co-cultures (MPCCs). MPCCs where infected with Gaussia-luciferase secreting HCV (see FIG. 2a) for 24 hours, washed 4 times and media replaced every 48 hours. Supernatants where collected at the indicated time-points and used to infect highly HCV permissive Huh7.5 cells. Positive staining for the HCV NS5a antigen (a marker for HCV infection) 72 hours following infection demonstrates that the supernatants contain infectious virus.

FIG. 6 depicts the results of treatment of microscale human liver tissues with trypsin/EDTA enhances infection with HCV-like particles. Infection of micropatterned co-cultures as in FIG. 2A, except some of the co-cultures were treated with 0.25% Trypsin/EDTA for 1 minute prior to incubation with viral inoculum (second column of pictures). Phase contrast micrographs are shown on top row, while GFP positive infected cells on the bottom row.

FIG. 7 depicts the development of HCV-dependent Fluorescence Relocalization (HDFR) reporter cells. IPS1 (also know as Cardif/MAVS/VISA) is a mitochondrial protein cleaved by HCV NS3/4A protease in the HCV infection pathway. Mitochondrially anchored IPS1/Cardif is cleaved and activated by HCV NS3/4A protease following sensing of viral dsRNA by RLH. Activated IPS1/Cardif, in turn, recruits appropriate IKKs to activate NF-kappaB and IRF, resulting in the induction of type I IFN in infected cells. See e.g., Meylan et al. Nature (2006) 442, 39-44. EGFP-IPS is a fragment of IPS/Cardif comprising the NS3/4A cleavage site, fused to EGFP

FIG. 8 is a schematic representation of EGFP-IPS and EGFP-IPS C508Y, a mutant that cannot be cleaved by HCV NS3/4A protease.

FIG. 9 depicts the Huh7.5 cell line stably expressing a modified red fluorescent version of EGFP-IPS (TagRFP-nls-IPS) and mito-EGFP. TagRFP-nls-IPS encodes TagRFP (a bright red fluorescent protein), a SV40 nuclear localization sequence (nls), and the mitochondrial targeting sequence from the C-terminus of IPS-1. Mito-EGFP encodes the mitochondrial targeting sequence from subunit VIII of cytochrome-c oxidase. HCVcc infection causes relocalization of the Tag-nls-IPS from the mitochondria to the nucleus as a result of cleavage by the HCV NS3/4A protease and subsequent transport to the nucleus via the nls. Mito-EGFP localization is unaltered by HCV infection and serves as a marker for the mitochondria. The TagRFP-nls-IPS stained mitochondria (light grey), Mito-EGFP stained mitochondria (dark grey), and TagRFP-nls-IPS localized to nucleus upon HCV infection (darkest grey) are depicted.

FIG. 10 depicts a flow diagram exemplifying a procedure which may be used to monitor or quantify the HCV infection of primary adult hepatocytes.

FIG. 11A depicts adult primary hepatocytes maintained by micropattern co-culture (MPCC) that express the HDFR cassette and were infected with HCVcc secreted gaussia reporter virus. MPCCs were first transduced with a lentivirus encoding either wt or C508Y mCherry-nls-IPS (similar to Tag-nls-IPS described above, using mCherry red fluorescent protein in place of TagRFP), and then infected with HCVcc reporter virus. After overnight incubation with HCVcc reporter virus, MPCCs transduced with wt mCherry-nls-IPS were treated with either DMSO or the HCV polymerase inhibitor 2′CMA. C508Y mCherry-nls-IPS transduced MPCCs were treated with DMSO. Relocalization of mCherry-nls-IPS from the mitochondria to the nucleus was observed in wt mCherry-nls-IPS transduced MPCCs treated with DMSO at 48 h post infection with HCVcc. Representative fluorescent images are shown.

FIG. 11B shows successful results measuring infected cells in DMSO-treated MPCCs, cultures treated with HCV polymerase inhibitor (2′CMA), and cells having the IPS-1 mutation (C508Y). The left graph shows quantification of nuclear mCherry fluorescence of MPCC islands infected with the HCVcc reporter virus plotted as the number of positive cells per island. The right graph shows gaussia luciferase activity in RLU (relative light units) detected in the cell culture supernatants at 48 h post infection of MPCC infected with HCVcc reporter virus.

FIG. 12 further depicts the infection of TagRFP-nls-IPS transduced MPCCs with HCVcc secreted gaussia reporter virus and serum from a HCV positive patient.

FIG. 13 depicts a flow chart of an exemplary cell-cell infection assay.

### DETAILED DESCRIPTION

The present invention features micropatterned co-culture systems having several uses in analyzing, diagnosing and treating viral diseases, in particular, infectious viral diseases such as hepatitis C virus (HCV), members of the Flavivirus genus, and malaria parasites such as Plasmodium falciparum, Plasmodium vivax, Plasmodium ovale, and Plasmodium malariae, and hepatitis A, B, δ, E.

In certain embodiments, the methods are used to determine key aspects of the mammalian, in particular, the human host response and/or propensity for chronic infection (e.g., chronic HCV infection). Understanding such aspects can further facilitate the identification and/or development of therapeutic compounds, in particular, new classes of therapeutic compounds that target specific pathways in the viral lifecycle.

Certain aspects of the invention feature cultures and systems, in particular, micropatterned co-cultured systems (e.g., micropatterned co-cultures of primary human hepatocytes and stromal cells) suitable as in vitro models of infectious diseases, in particular, infectious viral diseases such as Hepatitis C virus (HCV), virus serotypes of the genus Flavivirus, and malaria parasites such as Plasmodium falciparum, Plasmodium vivax, Plasmodium ovale, and Plasmodium malariae. The cultures and systems of the invention feature sustained and reproducible infection of hepatocytes, in particular human hepatocytes (e.g., isolated primary human hepatocytes in vitro). Infected cultures and/or systems of the invention preferably produce viral particles de novo and maintain normal levels of liver-specific functions (e.g., metabolic function) and, in particular, mimic physiologically-relevant host responses to viral infection. This is due, at least in part, to the fact the natural in vivo microenvironment of the hepatocytes (i.e., the cell-cell and cell-matrix communication environment) is maintained in the cultures and systems of the invention as are other key environmental features including, but not limited to, adequate diffusional environment (for delivery of nutrients and removal of waste products), adequate oxygen supply, and the like. For example, cells in living tissue are “polarized” with respect to diffusion gradients. Differential delivery of oxygen and nutrients, as occurs in vivo by means of the capillary system, controls the relative functions of tissue cells and their maturation. Thus, cell cultures and systems (e.g., bioreactors) of the invention simulate these in vivo delivery mechanisms and, accordingly, provide the necessary corollary to in vivo environments to measure natural responses in vitro. The cultures and/or systems of the invention feature hepatocytes (e.g., primary human hepatocytes) maintain their differentiated phenotypic function, are efficiently and reproducibly infected (high infectivity), and thus are extremely well suited to use in the analysis, diagnosis of viral disease and in the development of therapeutic treatments of same. Certain aspects of the invention further feature non-destructive methodologies for measuring and/or monitoring viral infection (e.g., viral replication, etc.). As such, the cultures and systems of the invention provide for effective monitoring of viral infection (e.g., HCV infection) in vitro, allowing for the development and testing of safe and effective HCV therapeutics. The cultures and systems of the invention further are advantageous in predicting in vivo liver metabolism and toxicity.

So that the invention may be more readily understood, certain terms are first defined.

An “immunogenic agent” or “immunogen” is capable of inducing an immunological response against itself on administration to a mammal, optionally in conjunction with an adjuvant.

The term “treatment” as used herein, is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the infection and the general state of the patient's own immune system.

The term “patient” includes human and other mammalian subjects that receive either prophylactic or therapeutic treatment.

As used herein and in the appended claims, the singular forms “a,” “and,” and “the” include plural referents unless the context clearly dictates otherwise. Thus, for example, reference to “a cellular island” includes a plurality of such cellular islands and reference to “the cell” includes reference to one or more cells known to those skilled in the art, and so forth.

### I. Uses

A. Screening Assays

In one aspect, the invention provides an in vitro model of infectious disease (e.g., hepatitis A, B, C, D, E, malaria, dengue, yellow fever, members of the Flavivirus genus) that can be utilized for identifying potential anti-infective agents and pharmaceutical drug development. The invention provides compositions, methods, and systems that allow development of viable, differentiated human hepatocyte cultures, co-cultured with accessory or supportive non-parenchymal cells in vitro with non-destructive optical readout of experimental results. The cultures and/or systems of the invention exhibit high infectivity which is believed to be crucial in the screening for anti-infective agents (e.g., anti-viral agents, anti-parasitic agents, etc.) Without wishing to be bound in theory, it is believed that the readout of an effective screening assay system should be on the order of at least 5-, 6-, 7-, 8-, 9-fold higher, preferably at least an order of magnitude higher for infected cells as compared to a suitable control (e.g., non-infected cultures/systems or infection-inhibited cultures/systems). This feature can be referred to as “high signal:noise” ratio.

The invention provides methods to increase the infectivity of cells in culture. The present invention shows that micropatterned co-cultured cells treated with trypsin/EDTA or EDTA alone show an increased rate of viral infection, and also a higher rate of viral replication (e.g., at least 2-, 3-, 4-, 5-, 6-, 7-, 8-, 9-, 10-fold higher than viral replication in untreated cultures).

The cultures and/or systems of the invention may be used in vitro to screen a wide variety of compounds, such as small molecules, antibodies, peptides, nucleic acid-based agents and the like, to identify agents that modify or inhibit viral or parasitic infection, replication, etc.

In a preferred embodiment these results may be assessed by observation of reporter gene fluorescence in the cell or in the media (e.g., when a reporter gene is released into the cytosol from the mitochondria upon viral infection, this may be observed as fluorescence in the cytosol by microscopy or automated optical readout).

In some embodiments, infectious diseases may be monitored in the presence and absence of test agents. For example, in liver cultures of the invention hepatitis B and C may be tested for their effects on heterotypic and homotypic interactions as well as interactions on particular cells. Furthermore, test agents used to treat such diseases may be studied. Similarly, malaria and other infectious diseases and potential therapeutics may be tested.

Biomarkers identified according to the methodologies of the invention (described in detail below), can also be used in screening assays for modulatory agents. This may also be accomplished using standard cytological and/or histological techniques including the use of immunocytochemical techniques employing antibodies recognize specific biomarkers. In this case, stable micropattern cultures may be exposed to a test agent. After incubation, the micropattern cultures may be examined for change in biomarker production (or procesins) as an indication of the efficacy of the test substance. Varying concentrations of the drug may be tested to derive a dose-response curve.

B. Target Validation

The cultures and/or systems (e.g., bioreactors) of the invention are particularly suited for use in target validation. In particular, the cultures and/or systems are useful for validating the predicted role of one or more biomolecules in the infection process. For example, proteins identified in preliminary studies (e.g., studies in conventional culture systems, studies in uninfected systems, differential expression studies, etc.) as playing a potential role in infectious processes or pathways can be tested in a culture or system of the invention to confirm the potential role. In certain embodiments, proteins identified from preliminary studies (proteins suspected to play a role in infectivity (e.g., cell entry receptors, etc.) are modulated (e.g., upregulated or downregulated) in cultures or systems of the invention and infectious activity is assayed following modulation. For example, candidate proteins can be “knocked down” using gene suppression techniques, for example, RNA interference (RNAi) as a means of downregulating. Inhibition of infectivity can be tested following downregulation and candidate proteins important in the infection process can be accordingly validated. The suitability of the cultures and/or systems of the invention derived particularly from the superior properties of the cultures/systems as in vitro models of virus or parasite infection including, but not limited to, the high infectivity of the system, the superior differentiated phenotype of the micropatterned hepatocytes, the ease and efficiency of the reporter system, and the like.

C. Biomarker Discovery

The cultures and/or systems (e.g., bioreactors) of the invention are particularly suited for use in biomarker discovery methodologies. In particular, biomarkers of infectious disease (e.g., diseases including, but not limited to, HCV, Plasmodium falciparum, Plasmodium vivax, Plasmodium ovale, Plasmodium malariae) can be identified by analyzing or studying cultures and/or systems of the invention to determine biomolecules (e.g., proteins, nucleic acids, etc) produced (e.g., expressed, for example, as mRNAs or proteins,) or processed (e.g., modified, activated, trafficked, secreted, etc.) by parenchymal and/or non-parenchymal cells included therein. Viral biomolecules can likewise be identified as biomarkers using the cultures and/or systems of the invention. In one embodiment, a biomarker of infection is identified in a method comparing biomolecules produced (or processed) in an infected (e.g., virally-infected) culture of the invention with those of a comparible non-infected culture/or system (or cell included therein) or to some other suitable control. A biomolecule whose production or processing (e.g., expression or activity, etc.) changes in the infected culture or system (or cell included therein) can thus be identified as a biomarker of the infected state. More than one changing biomarker can be evaluated simultaneously as a profile of the normal or infected state. The increase or decrease of biomarkers, for example, in samples being compared or within a sample being analyzed (e.g., proportional production or processing of one or more biomarkers) can be used as a profile. Useful profiles include, but are not limited to normal hepatocyte profiles (e.g., normal hepatocyte-produced markers), virally-infected hepatocyte profiles (including hepatocyte and/or virally-produced markers), and the like.

D. Biomarker-Based Diagnostics

In certain aspects, the identification of such biomarkers (and/or profiles) can aid in the diagnosis of infectious disease. In particular, the biomarkers (and/or profiles) can aid in the diagnosis of infectious disease in instances where traditional detection methodologies prove unsuccessful. For example, several infectious disease (e.g., viral or parasitic infections, for example, malaria, chronic HCV, etc.) have latent or indolent phases during which the infectious agent is not circulating in the bloodstream or is present at level below the limits of detection of standard diagnostic tests. In the case of chronic HCV, for example, circulating virus levels may be too low to produce detectable levels of anti-virus antibodies (antibodies produced by the host) or viral antigens (proteins produced by the virus, or nucleic acids encoding same) in blood or serum samples from the patient. Likewise, in cases of malaria, circulating parasite levels may be too low to detect microscopically or using immunochromatographic tests, or the infectious parasite may be masked from detection. The infectious agent can “hibernate” or lie dormant in the tissues of the patient, for example in the liver of the patient. Accordingly, a patient's blood work may appear normal but the infectious organism has not been irradiated or cleared. In such instances, infection-specific biomarkers (or profiles) identified using the cultures or systems of the invention can be used to detect latent or indolent infection and determine the course of treatment.

Biomarkers identified by analyzing or studying cultures and/or systems of the invention can be identified in virus- or parasite-infected cultures or systems. Biomarkers or profiles so-identified can be useful, either alone or in combination with normal cell profiles, in the diagnosis of infected patients and corresponding treatment thereof.

Several infectious diseases further have multiple genotypes or serotypes. In such diseases, biomarkers or profiles specific for the particular serotype/genotype can be used in the diagnosis and treatment of same (see below).

E. Monitoring Infection and/or Course of Treatment in Patients

The invention provides methods of monitoring patients suffering from infection, having latent infection, treated with one of more anti-viral agents, vaccines, etc. The methods can be used to monitor both therapeutic treatment on symptomatic patients and prophylactic treatment on asymptomatic patients.

Some methods entail determining a baseline value, for example, of an biomarker level or profile in a patient, before administering a dosage of agent, and comparing this with a value for the profile or level after treatment. A significant increase (i.e., greater than the typical margin of experimental error in repeat measurements of the same sample, expressed as one standard deviation from the mean of such measurements) in value of the level or profile signals a positive treatment outcome (i.e., that administration of the agent has achieved a desired response). If the value for immune response does not change significantly, or decreases, a negative treatment outcome is indicated.

In other methods, a control value (i.e., a mean and standard deviation) of level or profile is determined for a control population. Typically the individuals in the control population have not received prior treatment. Measured values of the level or profile in a patient after administering a therapeutic agent are then compared with the control value. A significant increase relative to the control value (e.g., greater than one standard deviation from the mean) signals a positive or sufficient treatment outcome. A lack of significant increase or a decrease signals a negative or insufficient treatment outcome. Administration of agent is generally continued while the level is increasing relative to the control value. As before, attainment of a plateau relative to control values is an indicator that the administration of treatment can be discontinued or reduced in dosage and/or frequency.

In other methods, a control value of the level or profile (e.g., a mean and standard deviation) is determined from a control population of individuals who have undergone treatment with a therapeutic agent and whose levels or profiles have plateaued in response to treatment. Measured values of levels or profiles in a patient are compared with the control value. If the measured level in a patient is not significantly different (e.g., more than one standard deviation) from the control value, treatment can be discontinued. If the level in a patient is significantly below the control value, continued administration of agent is warranted. If the level in the patient persists below the control value, then a change in treatment may be indicated.

In other methods, a patient who is not presently receiving treatment but has undergone a previous course of treatment is monitored for biomarker levels or profiles to determine whether a resumption of treatment is required. The measured level or profile in the patient can be compared with a value previously achieved in the patient after a previous course of treatment. A significant decrease relative to the previous measurement (i.e., greater than a typical margin of error in repeat measurements of the same sample) is an indication that treatment can be resumed. Alternatively, the value measured in a patient can be compared with a control value (mean plus standard deviation) determined in a population of patients after undergoing a course of treatment. Alternatively, the measured value in a patient can be compared with a control value in populations of prophylactically treated patients who remain free of symptoms of disease, or populations of therapeutically treated patients who show amelioration of disease characteristics. In all of these cases, a significant decrease relative to the control level (i.e., more than a standard deviation) is an indicator that treatment should be resumed in a patient.

The tissue sample for analysis is typically blood, plasma, serum, mucous fluid or cerebrospinal fluid from the patient. The sample is analyzed, for example, for levels or profiles of biomarkers. Biomarker levels or profiles can be determined by any art-recognized means, for example ELISA, RIA, Western blot immunodetection, Northern blot detection, etc. Biomarker levels of profiles can be determined in normal subjects or in subjects at risk for viral infection, e.g., as a baseline determination. Biomarker levels or profiles can be determined before and after treatment (e.g., anti-viral agent treatment, vaccine, etc), for example, at determined time intervals following treatment.

In some methods, a baseline measurement of biomarker levels or profiles in the patient is made before administration of a therapeutic treatment, a second measurement is made soon thereafter to determine the peak change in levels or profiles, and one or more further measurements are made at intervals to monitor for return of levels or profiles to pre-administration (e.g., infected) levels. Upon return of levels or profiles to pre-administration levels or profiles, return to baseline or a predetermined level or profile (e.g., a 50%, 25% or 10% change in level or one or more biomarkers), administration of further treatment may be warranted. In some methods, peak or subsequent measured levels or profiles (optionally corrected for background) are compared with reference levels or profiles previously determined to constitute a beneficial prophylactic or therapeutic treatment regime in other patients. If the measured level or profile is significantly changed as compared to a reference level (e.g., mean levels of one or more biomarkers for the reference population of patients plus or minus at least one standard deviation) additional treatment may be warranted.

Additional methods include monitoring, over the course of treatment, any art-recognized physiologic symptom (e.g., decreased appetite, fatigue, abdominal pain, jaundice, itching, flu-like symptoms, liver tissue inflammation, liver tissue scarring, abnormal results in liver function tests (e.g., elevation of alanine transaminase (ALT), aspartate transaminase (AST), and/or Gamma-glutamyl transferase (GGTP)), presence of virus in the blood or liver as measured by molecular biology techniques, presence of anti-viral antibodies in the patient) routinely relied on by researchers or physicians to diagnose or monitor infectious diseases (e.g., HCV). For example, one can monitor for the presence of anti-HCV antibodies to diagnose HCV or measure the level of ALT or AST to monitor HCV. The latter is a symptom of HCV but can also occur without other characteristics of either of these diseases.

Additional methods feature treatment of latent or asymptomatic patients. Latent disease may exhibit low viral loads and few or no symptoms. By contrast, individuals with asymptomatic disease may have high viral loads and experience a variety of possible disease conditions. In some cases disease progression is very slow, such that patients rarely or never experience significant disease symptoms. In other cases liver disease or cirrhosis develops slowly over a period of years.

F. Individualized Patient Regimes

The cultures and/or systems of the invention are particularly suited for use in individualized patient treatment regimes.

In one aspect, the cultures and/or systems of the invention are used to tailor a patient's treatment based on the specific serotype or genotype of the agent with which they are infected. For example, a patient's serum can be sampled and used for viral genotyping using the cultures/systems of the invention. In an exemplary embodiment, a blood sample is isolated from the patient (a blood draw, for example) and the sample, or virus (e.g., live virus) isolated thereform, is applied to a culture or system of the invention (comprising, for example, heterologous hepatocytes). The genotype of virus (or viruses) isolated from the patient is determined. Based on the genotype, the patient's treatment can be tailored with a genotype-specific regimen.

In another aspect, hepatocytes isolated from a patient are used in a culture or system of the invention. Hepatocytes can be isolated, for example, via liver biopsy. Alternatively, and perhaps preferred, hepatocytes can be derived from precursor cells isolated from the patient. For example, pluripotent stem cells can be isolated from the patient (e.g., from a skin biopsy from the patient) and induced in culture to differentiate into hepatocytes. Cultures and/or systems of the invention featuring patients-specific hapatocytes can be used in any on of a number of art-recognized individualized testing or treatment regimens.

G. Vaccine Discovery and Development

In addition to the above-described drug discovery methods, the cultures and/or systems of the invention are also particularly suited for vaccine discovery and development applications. Certain infectious diseases, for example malaria, progress through multiple stages or infection, certain of which are amenable to therapeutic treatment (using, for example, an anti-infective agent identified in a screening assay described herein) and certain of which are amenable to prophylactic treatment (e.g., vaccination). In developing infectious disease vaccines, attenuated viruses or attenuated parasites are often candidates for development. An art-recognized problem, however, is demonstrating that attenuation of the candidate organism is complete or total. The cultures and/or systems of the invention, in mimicking in vivo function, are particularly useful for demonstrating that, for example, a candidate attenuated infectious agent fails to “bud out” or traffic from hepatocytes (e.g., for entry into or infection of the bloodstream). Test vaccines can accordingly be validated in the cultures and/or systems of the invention. Candidate vaccine agents can include, but are not limited to genetic variants or organisms irradiated or otherwise subjected to attenuation means.

In some embodiments, the micropatterned co-culture systems may include immune cells which have been previously exposed to a disease antigen. In some preferred embodiments, the immune cells (e.g., B-cells or T-cells) are exposed to a disease antigen or potential vaccine antigens in vitro and are added to the co-culture. In other embodiments, the immune cells may be harvested from a patient having the disease, thereby extracting immune cells naturally exposed to the disease in vivo. In some embodiments, multiple immune cells may be incorporated into the cell-culture system (e.g., B-cells, Kupffer cells, macrophages, etc.). Accordingly, potential vaccines may be tested by exposing immune cells to a test vaccine composition and examining their ability to prevent infection or disease progression in the co-culture system.

In a related aspect the co-culture system of the invention may be employed to validate vaccines as described above. Disease progression may be measured by any means known in the art (e.g., progression to extra-hepatic stage may be observed as blebbing or bursting of hepatocytes in culture). In other embodiments, the co-culture system may include red blood cells, such that the infectivity of RBCs may be observed in culture.

H. Toxicity Studies

In addition to the above-described uses of the cultures and/or systems of the invention in the screening for anti-viral or anti-parasitic agents, the cultures and/or systems can further be used in toxicology studies to determine the toxicity of an agent identified an a potent anti-infective agent. Uninfected, or normal, cultures or systems are particularly suited to toxicology studies. Toxicology studies can be performed on cultures or systems featuring cells as the same type as used in the anti-infective agent screening assays described herein, or can comprise cells from a different source.

The cultures and/or systems of the invention may be used in vitro to test a variety of potential anti-infective compounds for their ability to cause cytotoxicity and/or death.

In a preferred embodiment these results may be assessed by observation of vital staining techniques, ELISA assays, immunohistochemistry, and the like or by analyzing the cellular content of the culture, e.g., by total cell counts, and differential cell counts or by metabolic markers such as MTT and XTT.

In one aspect of the invention, a stable, growing co-culture is established having a desired size (e.g., island size and distance between islands), morphology and may also include a desired oxygen gradient. The cells/tissue in the culture are exposed to varying concentrations of a test agent. After incubation with a test agent, the culture is examined to determine the highest tolerated dose—the concentration of test agent at which the earliest morphological abnormalities appear or are detected. Cytotoxicity testing may also be performed using a variety of supravital dyes to assess cell viability in the culture system, using techniques known to those skilled in the art. Once a testing range is established, varying concentrations of the test agent can be examined for their cytotoxic effect.

I. Multiple Tissue Studies

In certain aspects of the invention, the cultures and/or systems described herein are coupled or linked to one or more additional tissue culture systems modeling, for example, more complex in vivo processes. In exemplary embodiments, micropatterened hepatocyte co-cultures are coupled (e.g., in series) with a secondary tissue culture, the secondary culture being derived from a second tissue (e.g., a non-liver tissue) known to be involved in, for example, organ-organ interactions and/or hibernation of infectious diseases in vivo. In essence, the liver component of such a multi-culture system (modeled by the micropatterned hepatocyte co-cultures) can condition the infectious pathogen for infection or targeting of the secondary tissue (e.g., liver “conditioning” malaria prior to infection of the bloodstream). Cultures can be coupled or linked, for example, by fluidic circuits or by other culture device methodologies allowing containment of and communication between cultures.

Accordingly, the invention provides methods to study various stages of a pathogen life cycle by allowing the pathogen to infect one type of cell in culture, followed by infection of one or more additional cell types. This is particularly useful in situations where a pathogen must infect one cell type in order to progress to a phase wherein it has the ability to infect additional cell types. For example, the malaria parasite first infects the liver and subsequently infects red blood cells. The co-culture methods of the invention may be constructed such that the pathogen initially infects liver cells and then is allowed to infect red blood cells (RBCs). This may be achieved by, for example, including RBCs directly in the co-culture along with hepatocytes. Alternatively, virus produced in a co-culture not containing RBCs may be harvested and used to infect a second culture of RBCs. In another embodiment, two or more co-cultures may be connected using, e.g., a microfluidic channels such that pathogenic particles introduced to one co-culture may, after infection and reproduction, proceed to infect the second co-culture. Methods such as those described in the following publications, which are incorporated herein by reference, may be used in accordance with the co-cultures of the invention: U.S. Pat. Nos. 5,612,188; 7,288,405; U.S. patent application Ser. Nos. 11/436,100; 11/530,390; 11/533,322; 11/436,042; 11/533,331; 11/470,595; 11/530,381; 11/470,576; 11/533,607; 11/470,587

II. Micropatterned Co-Culture Systems

The invention primarily employs advanced micropatterned co-cultures (parenchymal cell-stromal cell cocultures) which, in some preferred embodiments, are combined with the reporter constructs and systems described below to yield powerful drug discovery and disease analysis platforms. Extensive descriptions of the technology related to the micropatterned co-cultures can be found in Khetani and Bhatia. Nature Biotechnology. 2008, 26(1):120-126; U.S. Pat. Nos. 6,133,030; 6,221,663; U.S. Pat. Pub. Nos. 20060270032 (patent application Ser. No. 11/440,289); 20060258000(patent application. Ser. No. 10/547,057); 20060160066 (patent application Ser. No. 11/336,131); 20050169962 (patent application Ser. No. 11/035,394); 20050008675 (patent application Ser. No. 10/750,293); and PCT Publication WO2006015368, all of which are incorporated herein by reference.

In some preferred embodiments the co-cultures of the invention are treated to increase the viral infectivity of the cells in culture or increase the rate of viral replication. The present invention shows that micropatterned co-cultured cells treated with trypsin/EDTA or EDTA alone show an increased rate of viral infection, and also a higher rate of viral replication (e.g., at least 2-, 3-, 4-, 5-, 6-, 7-, 8-, 9-, 10-fold or more higher than viral replication in untreated cultures). In some embodiments, the co-cultures are treated with trypsin/EDTA, EDTA, chemicals or compositions which disrupt cell-cell junctions, peptides/antibodies against claudin, peptides/antibodies/small molecules which disrupt tight junctions, gap junctions, or adherens junctions. In some embodiments, infectivity is increased by 2-, 3-, 4-, 5-, 6-, 7-, 8-, 9-, 10-fold or more. In some embodiments 0.00001%, 0.0001%, 0.001% 0.01%, 0.1%, 0.5%, 1%, 1.5%, 2%, 3%, 4%, 5%, 6%, 7%, 8%, 9%, 10%, 15%, 20%, 30%, 40% or more of the infectious particle (virus, bacteria, parasite, etc.) introduced into the culture subsequent to treatment initiate a cellular infection. Infectious particles can be viable, infection-competent particles. In certain embodiments, at least 0.01% of the infectious particles infect hepatocytes. In various embodiments, at least 1% of the infectious particles infect hepatocytes. Accordingly, in some embodiments, treatment of the culture according to the methods described herein may reduce the multiplicity of infection (MOI) required to generate co-cultures which model the disease of interest. In some embodiments the MOI is reduced by 2-, 3-, 4-, 5-, 6-, 7-, 8-, 9-, 10-, 20-, 30-, 40-, 50-, 100-, 200-, 500-fold or more.

The parenchymal cell-stromal cell cocultures utilize defined bounded geometries defining cell types. Using microfabrication tools, micropatterned configurations (from single cellular islands to large aggregates) outperform randomly distributed cocultures. Amongst the micropatterned configurations that were engineered, a balance of homotypic and heterotypic interactions can yield functional cocultures having defined or desired phenotypic activity, longevity and proliferative capacity.

In one aspect, micropatterned cultures comprising cellular islands of parenchymal cells and stromal cells are used. In this aspect, a substrate is modified and prepared such that stromal cells are interspersed with islands of parenchymal cells. Using microfabrication techniques modified, for example, from the semiconductor industry, the substrate is modified to provide for spatially arranging parenchymal cells (e.g., human hepatocytes) and supportive stromal cells (e.g., fibroblasts) in a miniaturizable format. The spatial arrangements can be a parenchymal cell type comprising a bounded geometric shape. The bounded geometric shape can be any shape (e.g., regular or irregular) having dimensions defined by the shape (e.g., diameter, width, length and the like). The dimensions will have a defined scale based upon their shape such that at least one distance from one side to a substantially opposite side is about 200-800 μm (e.g., where the shape is rectangular or oval, the distance between one side to an opposite side is 200-800 μm). For example, parenchymal cells (e.g., hepatocytes) can be prepared in circular islands of varying dimensions (e.g., 36 μm, 100 μm, 490 μm, 4.8 mm, and 12.6 mm in diameter; typically about 250-750 μm) surrounded by stromal cells (e.g., fibroblast such as murine 3T3 fibroblasts) or other materials. For example, hepatocyte detoxification functions are maximized at small patterns, synthetic ability at intermediate dimensions, while metabolic function and normal morphology were retained in all patterns.

In one embodiment, a bioreactor can use primary parenchymal cells (e.g., hepatocytes) alone or in combination with other cell types (other parenchymal and non-parenchymal cell types that can be used in the bioreactors and cultures systems of the disclosure include pancreatic cells (alpha, beta, gamma, delta), myocytes, enterocytes, renal epithelial cells and other kidney cells, brain cell (neurons, astrocytes, glia), respiratory epithelium, stem cells, and blood cells (e.g., erythrocytes and lymphocytes), adult and embryonic stem cells, blood-brain barrier cells, and other parenchymal cell types known in the art).

In one aspect, the reactor can be used with micropatterned parenchymal (e.g., hepatocytes) co-cultures and stromal cells (e.g., fibroblasts). The scale of the reactor can be altered to allow for the fabrication of a high-throughput microreactor array to allow for interrogation of xenobiotics. In one aspect, a microfluidic device is contemplated that has micropatterned culture areas in or along a fluid flow path.

As described herein the micropatterned cell island cultures of the invention are useful in drug discovery and development including screening for metabolic stability, drug-drug interactions, toxicity and infectious disease.

The cellular islands can take any geometric shape having a desired characteristic and can be defined by length/width, diameter and the like, based upon their geometric shape, which may be circular, oval, square, rectangular, triangular and the like. Furthermore, parenchymal cell function may be modified by altering the pattern configuration (e.g., the distance or geometry of the array of cellular islands). The distance between bounded geometric islands of cells may vary in a culture system (e.g., the distances between islands may be regular or irregular). The spatial distances between cellular islands may be random, regular or irregular. Furthermore, combinations of geometric bounded areas (e.g., cellular islands) of different geometries (e.g., multiple island sizes) may be present on a single substrate with varying distances (e.g., multiple island spacings) or regular distances between the islands.

In one aspect, the cellular islands comprise a diameter or width from about 250 μm to 750 μm. Similarly, where the geometric island comprises a rectangle, the width can comprise about 250 μm to 750 μm. In another aspect, the parenchymal cellular islands are spaced apart from one another by about 2 μm to 1300 μm from center to center of the cellular islands. In yet a further aspect, the parenchymal cell islands comprise a defined width (e.g., 250 μm to 750 μm) that can run the length of a culture area or a portion of the culture area. Parallel islands of parenchymal cells can be separated by parallel rows of stromal cells. In another aspect, the geometric shape may comprise a 3-D shape (e.g., a spheroid). In such instances, the diameter/width and the like, will be from about 250 μm to 750 μm.

As will be recognized in the art, the cellular islands may be present in any culture system including static and fluid flow reactor systems (e.g., microfluidic devices). Such microfluidic devices are useful in the rapid screening of agents where small flow rates and small reagent amounts are required.

The cellular culture of the invention can be made by any number of techniques that will be recognized in the art. For example, a method of making a plurality of cellular islands on a substrate can comprise spotting or layering an adherence material (or plurality of different cell specific adherence materials) on a substrate at spatially different locations each spot having a defined size (e.g., diameter) and spatial arrangement. The spots on the substrate are then contacted with a first cell population or a combination of cell types and cultured to generate cellular islands. Where difference cell-types are simultaneously contacted with the substrate, the substrate, coating or spots on the substrate will support cell-specific binding, thus providing distinct cellular domains. Methods for spotting adherence material (e.g., extracellular matrix material) can include, for example, robotic spotting techniques and lithographic techniques.

Various culture substrates can be used in the methods and systems of the invention. Such substrates include, but are not limited to, glass, polystyrene, polypropylene, stainless steel, silicon and the like. The choice of the substrate should be taken into account where spatially separated cellular islands are to be maintained. The cell culture surface can be chosen from any number of rigid or elastic supports. For example, cell culture material can comprise glass or polymer microscope slides. In some aspect, the substrate may be selected based upon a cell type's propensity to bind to the substrate.

The cell culture surface/substrate used in the methods and systems of the invention can be made of any material suitable for culturing mammalian cells. For example, the substrate can be a material that can be easily sterilized such as plastic or other artificial polymer material, so long as the material is biocompatible. A substrate can be any material that allows cells and/or tissue to adhere (or can be modified to allow cells and/or tissue to adhere or not adhere at select locations) and that allows cells and/or tissue to grow in one or more layers. Any number of materials can be used to form the substrate/surface, including, but not limited to, polyamides; polyesters; polystyrene; polypropylene; polyacrylates; polyvinyl compounds (e.g. polyvinylchloride); polycarbonate (PVC); polytetrafluoroethylene (PTFE); nitrocellulose; cotton; polyglycolic acid (PGA); cellulose; dextran; gelatin, glass, fluoropolymers, fluorinated ethylene propylene, polyvinylidene, polydimethylsiloxane, polystyrene, and silicon substrates (such as fused silica, polysilicon, or single silicon crystals), and the like. Also metals (gold, silver, titanium films) can be used.

As mentioned herein, in some instances the substrate may be modified to promote cellular adhesion and growth (e.g., coated with an adherence material). For example, a glass substrate may be treated with a protein (i.e., a peptide of at least two amino acids) such as collagen or fibronectin to assist cells in adhering to the substrate. In some embodiments, the proteinaceous material is used to define the location of a cellular island. The spot produced by the protein serves as a “template” for formation of the cellular island. Typically, a single protein will be adhered to the substrate, although two or more proteins may be used in certain embodiments. Proteins that are suitable for use in modifying a substrate to facilitate cell adhesion include proteins to which specific cell types adhere under cell culture conditions. For example, hepatocytes are known to bind to collagen. Therefore, collagen is well suited to facilitate binding of hepatocytes. Other suitable proteins include fibronectin, gelatin, collagen type IV, laminin, entactin, and other basement proteins, including glycosaminoglycans such as heparin sulfate. Combinations of such proteins also can be used.

The type of adherence material(s) (e.g., ECM materials, sugars, proteoglycans etc.) deposited in a spot will be determined, in part, by the cell type or types to be cultured. For example, ECM molecules found in the hepatic microenvironment are useful in culturing hepatocytes, the use of primary cells, and a fetal liver-specific reporter ES cell line. The liver has heterogeneous staining for collagen I, collagen III, collagen IV, laminin, and fibronectin. Hepatocytes display integrins .beta.1, .beta.2, .alpha.1, .alpha.2, .alpha.5, and the nonintegrin fibronectin receptor Agp110 in vivo. Cultured rat hepatocytes display integrins .alpha.1, .alpha.3, .alpha.5, .beta.1, and .alpha.6 .mu.1, and their expression is modulated by the culture conditions.

In particularly preferred embodiments, micropatterned hepatocyte co-cultures are employed. Due to species-specific differences in drug metabolism, human hepatocyte cultures can identify the metabolite profiles of drug candidates more effectively than non-human cultures.

The invention also provides methods of micropatterning useful to develop tissues with desired characteristics. A serial photolithographic based technique may be used (as described in U.S. Pat. Pub. No. 20060270032 and Khetani and Bhatia. Nature Biotechnology. 2008, 26(1):120-126, to create optimized micropatterned cocultures. The studies indicate that such cocultures can be miniaturized using stencil-based soft lithography in a multi-well format amenable for higher throughput experimentation. Patterning of various combinations and types of extracellular matrix proteins on a single substrate using robotic spotting techniques is a further method which may be employed. These matrix arrays coupled with parenchymal (e.g., hepatic) and stromal cocultures are amenable to high-throughput screening in drug development applications. The invention also provides functionally stable 2-D and 3-D cocultures in static and bioreactor settings with closed-loop flow conditions that approximate in vivo conditions. Furthermore, the micropatterning strategy can potentially be used to functionally optimize other systems in which cell-cell interactions are important (e.g., hematopoietic stem cells co-cultivated with stromal cell lines and keratinocytes with fibroblasts).

In general various printing and spotting techniques which may be used in the development of micropatterned co-cultures are described in U.S. Pat. Pub. No. 20060270032, incorporated herein by reference. In some embodiments, the spotting device comprises an apparatus and method like or similar to that described in U.S. Pat. Nos. 6,296,702, 6,440,217, 6,579,367, and 6,849,127, which are incorporated herein by reference.

With regard to placing insoluble and/or soluble factors at specific locations, various micro-spotting techniques using computer-controlled plotters or even ink-jet printers have been developed to spot such factors at defined locations. One technique loads glass fibers having multiple capillaries drilled through them with different materials loaded into each capillary tube. A substrate, such as a glass microscope slide, is then stamped out much like a rubber stamp on each glass slide. Spotting techniques involve the placement of materials at specific sites or regions using manual or automated techniques.

Conventional physical spotting techniques such as quills, pins, or micropipettors are able to deposit material on substrates in the range of 10 to 250 microns in diameter (e.g., about 100 spots/microwell of a 96 well culture plate). In some instances the density can be from 400 to 10000 spots per square centimeter, allowing for clearance between spots. Lithographic techniques, such as those provided by Affymetrix (e.g., U.S. Pat. No. 5,744,305, the disclosure of which is incorporated by reference herein) can produce spots down to about 10 microns square, resulting in approximately 800,000 spots per square centimeter.

A spotting device may employ one or more piezoelectric pumps, acoustic dispersion, liquid printers, micropiezo dispensers, or the like to deliver such reagents to a desired location on a substrate. In some embodiments, the spotting device comprises an apparatus and method like or similar to that described in U.S. Pat. Nos. 6,296,702, 6,440,217, 6,579,367, and 6,849,127.

Accordingly, an automated spotting device can be utilized, e.g. Perkin Elmer BioChip Arrayer™. A number of contact and non-contact microarray printers are available and may be used to dispense/print the soluble and/or insoluble materials on a substrate. For example, non-contact printers are available from Perkin Elmer (BioChip Arrayer™), Labcyte and IMTEK (TopSpot™), and Bioforce (Nanoarrayer™). These devices utilize various approaches to non-contact spotting, including piezo electric dispension; touchless acoustic transfer; en bloc printing from multiple microchannels; and the like. Other approaches include ink jet-based printing and microfluidic platforms. Contact printers are commercially available from TeleChem International (Arraylt™).

Non-contact printing will typically be used for the production of cellular microarrays comprising cellular islands. By utilizing a printer that does not physically contact the surface of substrate, no aberrations or deformities are introduced onto the substrate surface, thereby preventing uneven or aberrant cellular capture at the site of the spotted material.

Printing methods of interest, including those utilizing acoustic or other touchless transfer, also provide benefits of avoiding clogging of the printer aperature, e.g. where solutions have high viscosity, concentration and/or tackiness. Touchless transfer printing also relieves the deadspace inherent to many systems. The use of print heads with multiple ports and the capacity for flexible adjustment of spot size can be used for high-throughput preparation.

The total number of spots on the substrate will vary depending on the substrate size, the size of a desired cellular island, and the spacing between cellular islands. Generally, the pattern present on the surface of the support will comprise at least 2 distinct spots, usually about 10 distinct spots, and more usually about 100 distinct spots, where the number of spots can be as high as 50,000 or higher. Typically, the spot will usually have an overall circular dimension (although other geometries such as spheroids, rectangles, squares and the like may be used) and the diameter will range from about 10 to 5000 μm (e.g., about 200 to 800 μm).

By dispensing or printing onto the surfaces of multi-well culture plates, one can combine the advantages of the array approach with those of the multi-well approach. Typically, the separation between tips in standard spotting device is compatible with both a 384 well and 96 well plates; one can simultaneously print each load in several wells. Printing into wells can be done using both contact and non-contact technology.

The invention can utilize robotic spotting technology to develop a robust, accessible method for forming cellular microarrays or islands of a defined size and spatial configuration on, for example, a cell culture substrate. As used herein, the term “microarray” refers to a plurality of addressed or addressable locations.

In one aspect, the invention provides methods and systems comprising a modified printing buffer used in a spotting device to allow for ECM deposition, and identifying microarray substrates that permit ECM immobilization. The methods and systems of the invention are useful for spotting substantially purified or mixtures of biological proteins, nucleic acids and the like (e.g., collagen I, collagen III, collagen IV, laminin, and fibronectin) in various combinations on a standard cell culture substrate (e.g., a microscope slide) using off-the-shelf chemicals and a conventional DNA robotic spotter.

In another aspect, the invention utilizes photolithographic techniques to generate cellular islands. Drawing on photolithographic micropatterning techniques to manipulate functions of rodent hepatocytes upon co-cultivation with stromal cells, a microtechnology-based process utilizing elastomeric stencils to miniaturize and characterize human liver tissue in an industry-standard multiwell format was used. The approach incorporates ‘soft lithography,’ a set of techniques utilizing reusable, elastomeric, polymer (Polydimethylsiloxane-PDMS) molds of microfabricated structures to overcome limitations of photolithography. In one aspect, the invention provides a process using PDMS stencils consisting of 300 μm thick membranes with through-holes at the bottom of each well in a 24-well mold. To micropattern all wells simultaneously, the assembly was sealed against a polystyrene plate. Collagen-I was physisorbed to exposed polystyrene, the stencil was removed, and a 24-well PDMS ‘blank’ was applied. Co-cultures were ‘micropatterned’ by selective adhesion of human hepatocytes to collagenous domains, which were then surrounded by supportive murine 3T3-J2 fibroblasts. The size (e.g., geometric dimension) of through-holes determined the size of collagenous domains and thereby the balance of homotypic (hepatocyte/hepatocyte) and heterotypic (hepatocyte/stroma) interactions in the microscale tissue. Similar techniques can be used to culture cellular islands of other parenchymal cell types.

In another aspect of the invention techniques are used to generate micropatterns of biomolecules and/or cells on standard laboratory materials through selective ablation of a physisorbed biomolecule with oxygen plasma. In certain embodiments oxygen plasma is able to ablate selectively physisorbed layers of biomolecules (e.g., type-I collagen, fibronectin, laminin, Matrigel) along complex, non-linear paths which are difficult or impossible to pattern using alternative methods. In addition, certain embodiments relate to the micropatterning of multiple cell types on curved surfaces, multiwell plates, and flat-bottom flasks. The techniques and methods to achieve such micropatterning in accordance with the methods of the present invention are fully described in U.S. patent application Ser. No. 11/974,341, which is incorporated herein by reference in its entirety.

The term “adherence material” is a material deposited on a substrate or chip to which a cell or microorganism has some affinity, such as a binding agent. The material can be deposited in a domain or “spot”. The material and a cell or microorganism interact through any means including, for example, electrostatic or hydrophobic interactions, covalent binding or ionic attachment. The material may include, but is not limited to, antibodies, proteins, peptides, nucleic acids, peptide aptamers, nucleic acid aptamers, sugars, proteoglycans, or cellular receptors.

A cellular island or “spot” refers to a bounded geometrically defined shape of a substantially homogenous cell-type having a defined border. In one aspect, the cellular island or spot is surrounded by different cell-types, materials (e.g., extracellular matrix materials) and the like. The cellular islands can range in size and shape (e.g., may be of uniform dimensions or non-uniform dimensions). Cellular islands may be of different shapes on the same substrate. Furthermore, the distance between two or more cellular islands can be designed using methods known in the art (e.g., lithographic methods and spotting techniques). The distances between cellular islands can be random, regular or irregular. The distance between and/or size of the cellular islands can be modified to provide a desired phenotypic characteristic of morphology to a particular cell types (e.g., a parenchymal cell such as a hepatocyte).

In addition to modulating cellular islands to control heterotypic and/or homotypic interactions the invention can use a bioreactor system that provides the ability to modulate oxygen and nutrient uptake processes of mammalian cells to create a directional gradient in a reactor system. Directional oxygen gradients are present in various biological environments such as, for example, in cancer, tissue development, tissue regeneration, wound healing and in normal tissues. As a result of oxygen gradients along the length of a bioreactor system result in cells exhibiting different functional characteristics based on local oxygen availability. Accordingly, the invention provides methods, reactor systems and compositions that provide the ability of develop human tissues in vitro characteristic of normal tissue, but also to provide similar physiological environments by mimicking oxygen and/or nutrient gradients found in tissues in the body.

The use of the micropattern technology in combination with a bioreactor system allows for the development of microarray bioreactors. The bioreactor utilizes co-cultures of cells in which at least two types of cells are configured in a bounded geometric pattern on a substrate. Such micropatterning techniques are useful to modulate the extent of heterotypic and homotypic cell-cell contacts. In addition, co-cultures have improved stability and thereby allow chronic testing (e.g., chronic toxicity testing as required by the Food and Drug Administration for new compounds).

Typically, in practicing the methods of the disclosure, the cells are mammalian cells, although the cells may be from two different species (e.g., pigs, humans, rats, mice, and the like). It is understood that the methods and systems described herein may be applicable to the investigation and treatment of various animal diseases (e.g., the culturing of porcine hepatocytes and the treatment of porcine diseases, and so forth). The cells can be primary cells, or they may be derived from an established cell-line. Although any cell type that adheres to a substrate can be used in the methods and systems of the disclosure (e.g., parenchymal and/or stromal cells), exemplary combinations of cells for producing the co-culture include, without limitation: (a) human hepatocytes (e.g., primary hepatocytes) and fibroblasts (e.g., normal or transformed fibroblasts, such as NIH 3T3-J2 cells); (b) hepatocytes and at least one other cell type, particularly liver cells, such as Kupffer cells, Ito cells, endothelial cells, and biliary ductal cells; and (c) stem cells (e.g., liver progenitor cells, oval cells, hematopoietic stem cells, embryonic stem cells, and the like) and human hepatocytes and/or other liver cells and a stromal cell (e.g., a fibroblast). Other combinations of hepatocytes, liver cells, and liver precursor cells may be used. In some embodiments it may be desirable to include immune cells in the co-culture, e.g., Kupffer cells, macrophages, B-cells, dendridic cells, etc. Hepatocytes which may be cultured in the co-culture system as described herein may be from any source known in the art, e.g., primary hepatocytes, progenitor-derived, ES-derived, induced pluripotent stem cells (iPS-derived), etc. Hepatocytes useful with the present invention may be produced by the methods described in Takashi Aoi et al., Science 321 (5889): 699-702; U.S. Pat. Nos. 5,030,105; 4,914,032; 6,017,760; 5,112,757; 6,506,574; 7,186,553; 5,521,076; 5,942,436; 5,580,776; 6,458,589; 5,532,156; 5,869,243; 5,529,920; 6,136,600; 5,665,589; 5,759,765; 6,004,810; U.S. patent application Ser. Nos. 11/663,091; 11/334,392; 11/732,797; 10/810,311; and PCT application PCT/JP2006/306783, all of which are incorporated herein by reference in their entirety.

Further cell types which may be cultured in the co-culture system (as patterned or unpatterned cells) described herein include pancreatic cells (alpha, beta, gamma, delta), enterocytes, renal epithelial cells, astrocytes, muscle cells, brain cells, neurons, glia cells, respiratory epithelial cells, lymphocytes, erythrocytes, blood-brain barrier cells, kidney cells, cancer cells, normal or transformed fibroblasts, liver progenitor cells, oval cells, adipocytes, osteoblasts, osteoclasts, myoblasts, beta-pancreatic islets cells, stem cells (e.g., embryonic stem cells, hematopoietic stem cells, mesenchymal stem cells, endothelial stem cells, etc.), cells described in U.S. patent application Ser. No. 10/547,057 paragraphs 0066-0075 which is incorporated herein by reference, myocytes, keratinocytes, and indeed any cell type that adheres to a substrate.

It is understood that the micropatterned co-culture may contain parenchymal cells with one, or two or more types of non-parenchymal cells such as, for example, stromal cells, endothelial cells, stellate cells, cholangiocytes (bile duct cells), Kupffer cells, pit cells, etc. In some embodiments, the parenchymal cells (e.g., hepatocytes) are patterned (e.g., in spots), and the non-parenchymal cells are not patterned (e.g., they are mixed and distributed around the spotted parenchymal cells. In some embodiments, the cell culture may contain at least one non-parenchymal cell population. In certain embodiments, the cell culture may contain more than one non-parenchymal cell population. One of skill in the art will appreciate that particular patterns of non-parenchymal cells surrounding the parenchymal cells may be desired in some cases, e.g., when it is desired to mimic certain in vivo environments. It is understood that any support or accessory cells may be included in the micropatterned co-culture method of the invention.

In another aspect, certain cell types have intrinsic attachment capabilities, thus eliminating a need for the addition of serum or exogenous attachment factors. Some cell types will attach to electrically charged cell culture substrates and will adhere to the substrate via cell surface proteins and by secretion of extracellular matrix molecules. Fibroblasts are an example of one cell type that will attach to cell culture substrates under these conditions.

Cells useful in the methods of the disclosure are available from a number of sources including commercial sources. For example, hepatocytes may be isolated by conventional methods (Berry and Friend, 1969, J. Cell Biol. 43:506-520) which can be adapted for human liver biopsy or autopsy material. In general, cells may be obtained by perfusion methods or other methods known in the art, such as those described in U.S. Pat. Pub. No. 20060270032.

### 3-Dimensional Co-Cultures

In another aspect the co-cultures may be produced in a 3-dimensional scaffold. The invention provides methods for 3-dimensional patterning of cells within a 3-dimensional scaffold by providing a photopolymerization method for the formation of a hydrogel scaffold with the desired 3-dimensional structure or by patterning the cells within hydrogel scaffold by dielectrophoresis (DEP). DEP can be used alone for patterning of cells in relatively homogeneous slabs of hydrogel or in conjunction with the photopolymerization method. The methods allow for the formation of three dimensional scaffolds from hundreds of microns to tens of centimeters in length and width, and tens of microns to hundreds of microns in height. A resolution of up to 100 microns in the photopolymerization methods and possible single cell resolution (10 micron) in the DEP method is achievable.

Biopolymers suitable for use with the 3-D co-cultures include any polymer that is gellable in situ, i.e. one that does not require chemicals or conditions (e.g. temperature, pH) that are not cytocompatible. This includes both stable and biodegradable biopolymers. The photolithography method requires the use of polymers in which polymerization can be promoted by exposure to an appropriate wavelength of light (i.e. photopolymerizable) or a polymer which is weakened or rendered soluble by light exposure or other stimulus. The DEP method preferably uses a photopolymerizable polymer; however, any polymer with activatable or sufficiently slow polymerization kinetics to allow for patterning of the cells before polymerization can be used in the DEP method of the invention. Polymers that can be used in the methods of the invention include, but are not limited to, PEG hydrogels, alginate, agarose, collagen, hyaluronic acid (HA), peptide-based self-assembling gels, thermo-responsive poly(NIPAAm). Although some of the polymers listed are not innately light sensitive (e.g. collagen, HA), they may be made light sensitive by the addition of acrylate or other photosensitive groups. A number of biopolymers are known to those skilled in the art (Bryant and Anseth, 2001; Mann et al., 2001; and Peppas et al., 2000; all incorporated by reference). As the development of biopolymers is ongoing, it is understood that the exact selection of biopolymer for use is not a limitation of the invention. Any cytocompatible polymer with the appropriate polymerization properties can be used in the invention. The selection of appropriate polymers is well within the ability of those skilled in the art. The biopolymers may additionally contain any of a number of growth factors, adhesion molecules, degradation sites or bioactive agents to enhance cell viability or for any of a number of other reasons. Such molecules are well known to those skilled in the art.

A photoinitiator is a molecule that is capable of promoting polymerization of hydrogels upon exposure to an appropriate wavelength of light as defined by the reactive groups on the molecule. In the context of the invention, photoinitiators are cytocompatible. A number of photoinitiators are known that can be used with different wavelengths of light. For example, 2,2-dimethoxy-2-phenyl-acetophenone, HPK 1-hydroxycyclohexyl-phenyl ketone and Irgacure 2959 (hydroxyl-1-[4-(hydroxyethoxy)phenyl]-2methyl-1propanone) are all activated with UV light (365 nm). Other crosslinking agents activated by wavelengths of light that are cytocompatible (e.g. blue light) can also be used with the method of the invention.

Patterned cells of the invention may be localized in specked locations that may occur in repeating structures within 3-dimensional biopolymer rather than being randomly localized throughout 3-dimensional slab of biopolymer, on the surface of a regularly or irregularly shaped 3-dimensional scaffold, or patterned on a 2-dimensional support (e.g. on a glass slide). The cells can be patterned by locating the cells within specific regions of relatively homogeneous slabs of biopolymers (resolution up to about 5 microns) or by creating patterned biopolymer scaffolds of defined patterns wherein the living cells are contained within the hydrogel (resolution up to about 100 microns). Patterning is performed without direct, mechanical manipulation or physical contact and without relying on active cellular processes such as adhesion of the cells. This substantially increases the number of cells that can be efficiently patterned in a short period of time (minutes and increases patterning efficiency (number of patterned cells/total cells)). The cells are patterned by selective polymerization of the biopolymer or by patterning of the cells using an electrical field or both. Theoretically a single cell can be patterned by locating it in a specific position within a biopolymer; however, it is preferred that a plurality of cells, at least 10, preferably at least 20, more preferably at least 100, most preferably at least 500 cells, are patterned. Patterning does not require localization of all cells to a single, discrete location within the biopolymer. Cells can be localized, in lines one or two or many cells wide, or in multiple small clusters throughout a relatively homogeneous biopolymer scaffold (e.g. approximately 20,000 clusters of 10 cells each in a single scaffold). The 3-dimensional patterning can also include patterning of cells or other particles in a single plane by DEP as the cells are contained in a three dimensional scaffold. This is distinct from patterning of cells on a glass slide as the cells are contacted on all sides by the biopolymer. The cell patterning methods can also be used for patterning of organelles, liposomes, beads and other particles.

Relatively homogeneous slab of biopolymer refers to a polymerized biopolymer scaffold that is approximately the same thickness throughout and is essentially the same shape of the casting or DEP chamber in which it was polymerized.

Patterned biopolymer scaffold refers to a biopolymer scaffold that is of a substantially different shape than the casting or DEP chamber in which it was polymerized. The pattern could be in the form of shapes (e.g. circles, stars, triangles) or a mesh or other form. In one embodiment, the biopolymer is patterned to mimic in vivo tissue architecture, such as branching structures.

The methods allow photopolymerization of biopolymers of various heights that can be patterned using masks, preferably emulsion masks. Photopolymerization apparatus, DEP apparatus, and other methods to produce the 3-dimensional co-cultures of the invention are described in U.S. patent application Ser. No. 11/035,394, which is incorporated herein by reference.

The 3-D co-culture method can be used for the production of any of a number of patterns in single or multiple layers including geometric shapes or a repeating series of dots with the features in various sizes. Alternatively, multilayer biopolymer gels can be generated using a single mask turned in various orientations. The formation of high resolution patterned cells in 3-dimensions can be achieved by methods other than photopolymerization, such that the limitations of the method are overcome.

The ability to pattern individual cells provides a method for understanding cell-cell interactions in both 2- and 3-dimensions in tissues. Articular cartilage, for example, has distinct zonal layers: at the articular surface, disk-shaped chondrocytes produce a parallel collagen network and secrete lubricating molecules, whereas deep zone cells are organized into multicellular columnar lacunae surrounded by collagen fibers oriented perpendicular to the surface to resist compressive loads. Understanding the effect of cell shape and cell contact on matrix content and structure (and vice versa) is important for improving tissue-engineered constructs and elucidating the causes and development of osteoarthritis, a progressive and irreversible tissue degeneration. Chondrocytes for use in the instant invention for the study of cells within patterned scaffolds and the use of scaffolds for tissue replacement include primary cells from human, pig or other animal source, immortalized primary cell lines, chondrocyte cell lines, adult stem cell derived chondrocytes (i.e. from oval cells, bone marrow cells) embryonic stem cells and fetal chondrocytes. Furthermore, chondrocytes require a rounded morphology for differentiated function and are therefore not amenable to current 2-dimensional patterning methods. Recent literature suggests that cell seeding density in random 3-dimensional gel culture influences chondrocyte proliferation, gene expression patterns, and quantity and types of matrix molecule secretion, in a complex manner. In these reports, varying cell density affects several factors: nutrient exchange, cell-cell vs. cell-matrix contact, and cell proximity via paracrine signals. To isolate specific microenvironmental effects, chondrocytes can be positioned into 3-dimensional patterns designed to independently specify cell-cell contacts, cell proximity and/or cell shape, while providing constant nutrient exchange, total cell number, and average seeding density. For example, cell clusters of various sizes can be generated to isolate effects cell-cell contact or paracrine signaling, increasing with cluster size. Separating these effects is achieved by patterning cells with an attached pericellular matrix (few microns thick) to prevent physical contacts. Functional assays for matrix production (immunohistochemistry for sulfated GAG and Collagen II), zone-specific proteins (SZP for superficial layer, CILP for mid zone), and proliferation will determine cellular responses for various chondrocyte sources, including mesenchymal stem cells that may differentiate into zonal phenotypes.

Tissues with cell-derived anisotropic properties can be made using the DEP method disclosed herein. These patterned cells in scaffolds are useful for engineering tissues such as cartilage, tendon, and muscle. Chondrocytes, fibroblasts, or myocytes in patterned lines should exhibit anisotropic mechanical properties due to cell orientation and local deposition of matrix molecules.

It is understood that such 3-D micropatterned co-cultures may also be infected with pathogens for drug discovery, vaccine development, and other uses identified herein with regard to the micropatterned co-cultures in general. As such, all description and uses discussed herein apply, in particular embodiments, to 3-D micropatterned co-cultures.

Furthermore, 3-D co-cultures offer unique opportunities for analysis of infectious disease. Such cultures developed as 3D models of human tissue may be implanted in animals and therefore serve as “humanized” animal responses, where it is expected that the animal implanted with human tissue culture may respond to a disease in a way that is more similar to a human. In such embodiments, the animal may be infected with a pathogen (e.g., HCV, malaria, etc.) such that the disease may be more easily monitored or various treatments or drugs may be tested in the animal model.

### III. Reporter Systems

The present invention further employs a reporter construct and system which, when used in concert with the micropatterned co-culture system described above, may be used to develop a powerful platform to investigate infections diseases such as HCV, malaria, dengue fever, and others for which reliable and stable culture systems are not currently available.

In some aspects, the invention provides methods to conduct high throughput screening of micropatterned co-cultures which are infected with parasites, protozoans, bacteria, or viral particles, e.g., HCV, Plasmodium falciparum, Plasmodium vivax, Plasmodium ovale, Plasmodium malariae, virus serotypes of the genus Flavivirus, Dengue virus, etc. Although the micropatterned co-culture system provides a high throughput system for culture investigation (e.g., by employing 24 well, 96 well, 384 well plates, among other methods), traditional assay methods may impede analysis in certain disease systems. For example, in studies of viral infectivity, virus production is commonly measured by PCR, which requires harvesting and destruction of the culture cells. Advanced reporter constructs allow automated optical readout of viral replication, increasing the efficiency of viral culture studies without necessarily destroying culture cells.

Reporter constructs which may be used in accordance with the present invention are constructs similar to that disclosed in Jones et al. Journal of Virology. 2007, 81(16):8374-8383 which is incorporated herein by reference. The construct of Jones et al. is a modified HCV genome containing the reporter Renilla luciferase gene inserted between the p7 and NS2 coding sequences of HCV. In this construct, the signal peptide of p7 directs the reporter to the ER lumen. It is known that the measurement of luciferase activity may be used as a sensitive measure of rival RNA replication.

In one embodiment, viral particles containing the construct (i.e., modified reporter genome) of Jones et al. Journal of Virology. 2007, 81(16):8374-8383 is employed in the present invention to infect micropatterned hepatocyte co-cultures, thereby producing HCV infected cells in culture. In such embodiments, the micropatterned hepatocyte co-cultures are infected with viral particles containing the modified viral genome (e.g., that of Jones et al.). Subsequent intracellular viral replication yields a concomitant production of reporter production, which can be measured as a change in fluorescence intensity, e.g., an increase in fluorescence in the media or cytosol (depending upon vector design). One of skill in the art will appreciate that a similar strategy may be employed with other infective agents, e.g., hepatitis A, B, C, δ and E viruses, dengue virus, etc.

Modified versions of the construct may also be employed, for example, by varying the type or location of the fluorescent reporter protein in the construct, by changing the signal peptide, or by using fragments of parasite/viral sequence rather than the entire genome. Locations of the reporter could be at any site in the genome that is compatible with virus replication (RNA replication and virus production). For example, for HCV, the reporter may be inserted in domain III of NS5A, in-frame but upstream of the complete HCV polyprotein, or in the context of biscistronic viruses where the reporter is positioned in either the first of the second cistron. In addition it is also possible to employ single cycle infectious particles that are produced by trans-complementation of the structural proteins, e.g. constructs similar to those in J Virol. 2008 July; 82(14):7034-46 which is incorporated herein by reference. A skilled artisan will appreciate that other reporter systems not specifically mentioned but known in the art can be used. For example, luminescence and/or fluorescence based reporter systems that can exploit a biological interaction such as cleavage of a viral protein such as NS3/4a with a host protein such as IPS1 can be used.

In some embodiments a reporter construct or vector may be introduced into the cell culture before infection with a disease causing agent. This may be useful, for example, when it is desired to introduce a reporter construct which indicates infection rather than replication.

In a related embodiment, using a first vector, a mitochondrial localization signal (MLS) may be used to direct the fluorescent reporter to the mitochondrion in uninfected cells. Such cells would show a “spotted” fluorescence wherein the cells exhibit fluorescence in the mitochondria but not the cytoplasm. Upon infection with a virus and subsequent cleavage of the reporter protein from the signal sequence, the fluorescent protein is released into the cytoplasm, allowing visualization of fluorescence throughout the cell.

It was discovered that a mitochondially localized adaptor protein, called (IPS-1 (Cardif, MAVS, VISA) has a site for the HCV NS3-4A serine proteinase. The NS3-4A serine proteinase is targeted to the mitochondrion and cleaves this adaptor liberating its cytosolic domain. Typically this blocks the RIG-I pathway which senses pathogen associated molecular patterns (such as, e.g., 5′ triphosphate, oligo A, or oligo U, and dsRNA). The present invention provides vectors and compositions facilitating the replacement the cytosolic domain of IPS-1 with a fluorescent reporter (e.g., GFP). The resulting fusion protein becomes localized to mitochondria, and such cells show a “spotted” fluorescence wherein the cells exhibit fluorescence in the mitochondria but not the cytoplasm. Upon infection of the cell with HCV, the virus, replicates and produces NS3-4A which cleaves IPS-1, releasing the reporter into the cytosol allowing visualization of fluorescence throughout the cell. This change in fluorescence can be detected and quantified by IF (microscopy or Imagestream). Such a reporter has been reduced to practice by transduction into primary hepatocytes using a lentivirus vector, which was then seeded into microscale co-cultures. Because the co-cultures may be made in high-throughput formats, the ability to detect infectivity using standard optical methods (described further below) allows arrays of such cultures to be analyzed quickly and efficiently. In is understood that such reporter constructs, tailored to other infectious pathogens (e.g., Hepatitis A, B, D, E, dengue, and others mentioned herein) will be useful for monitoring pathogen infectivity.

In other embodiments relocalization may not be from the mitochondria to the cytosol, but from the mitochondria to other cellular compartments, e.g., the nucleus. In one embodiment, an HCV-dependent fluorescence relocalization (HDFR) cassette (see Example 2) is employed that comprises a fluorescent protein (e.g. EGFP, mCherry or TagRFP, etc.), a nuclear localization sequence (e.g., an SV40 nuclear localization sequence (NLS)), and a mitochondrial-targeting domain (e.g., a mitochondrial targeting protein derived from the interferon-beta promoter stimulator 1 protein, IPS-1). See Examples 2, 3, and 4 for further embodiments of reporter constructs and methods of the present invention. One of skill in the art will appreciate that arrangements of similar components that relocalize fluorescence to the nucleus or other cellular compartments may be employed.

Such embodiments may be particularly useful for monitoring the infectivity of viruses or parasites. For example, the efficacy of a drug may be monitored by introducing the infectious agent (malaria parasite, HCV, or a Flavivirus, etc.) into a micropatterned co-culture along with said drug, whereby the drug is seen to be effective in preventing infection if a change in fluorescence is not observed (e.g., cytosolic fluorescence).

In some embodiments, the pathogen may be unmodified and/or not include a reporter construct. As such the pathogens may be detected by measuring their replication by convention methods known in the art (e.g., PCR, RT-PCR, immunostaining, Western Blot, etc.).

One of skill in the art will appreciate that reporter genes other than Renilla luciferase may be used in the modified viral genomes, e.g., variants of Firefly luciferase, Gaussia luciferase, SEAP, Green Fluorescent Protein, variants of Yellow Fluorescent Protein, and variants of Red Fluorescent Protein, mCherry, tdTomato, etc. Any of the fluorescent proteins, reporter constructs, and vectors described in Examples 1-4 may be employed, e.g., the mCherry-based HDFR cassette, the mCherry-nls-IPS construct, the Tag-nls-IPS construct, etc.

A further difficulty with attempting to infect cell cultures with viral particles is that infection is often very low. Thus, in order for high throughput screening and analysis to be performed, it is important to employ methods which increase the infectivity of the viral particles. The present inventors have surprisingly discovered that exposure of the micropatterned co-cultures to trypsin/EDTA or EDTA dramatically improves access to viral pseudoparticles and virus. The resultant increases in infection efficiency (and improved signal to noise) are vital for many applications in drug screening, vaccine development, and further diagnostic applications. Indeed, such treated co-cultures were unexpectedly found to be more infectable than fresh human hepatocytes.

It will be appreciated by the skilled artisan that the reporter systems described herein are useful not only for HCV infection in micorpatterned co-culture systems, but in many other cells and cell culture systems, e.g., any cell culture featuring cells susceptible to HCV infection, or, in embodiments wherein pathogens other than HCV are being studied, any cell culture featuring cells susceptible to infection by the pathogen of interest.

Because the micropatterned co-culture system of the invention may be in a high-throughput format (microarray formats, 384+ well plates), cultures of the invention transfected or infected with reporter constructs (e.g., modified viral particles) may be analyzed easily using optical recognition systems. In such embodiments, fluorescent reporter proteins may be detected in an automated fashion using techniques known in the art, e.g., cameras and image recognition technology. Techniques for detecting fluorescence are described in Wells. Current Opinion in Biotechnology. 2006, 17(1):28-33 and in U.S. Pat. Nos. 6,416,959; 6,716,588; 6,548,263; 6,986,993; 6,756,207; 6,759,206; 6,653,124; 6,103,479; 6,902,883; 6,727,071; and U.S. patent application Ser. No. 11/809,937, which are all incorporated herein by reference.

In other embodiments the invention may be optimized for high-content, low-throughput studies. For example versions of the platform may be scaled up, e.g., a micropatterned T-75 flask, if a larger cell number is needed. In smaller scale experiments, analysis may be performed by more conventional methods to evaluate anti-disease agents e.g., PCR, RT-PCR, immunostaining, Western Blot, etc.

### IV. Infectious Diseases

The methods and compositions of the present invention are applicable to a wide range of viral and non-viral infectious diseases. In the most preferred embodiments, the infectious diseases are diseases which infect liver cells or exist in the liver during a portion of their infection or life cycle. Information is provided below for several preferred infectious diseases, although this is not to be considered a limiting group. The methods and compositions of the invention may be applied generally to, for example, all hepatitis viruses (e.g., hepatitis C virus (HCV)), virus serotypes of the genus Flavivirus, and malaria parasites such as Plasmodium falciparum, Plasmodium vivax, Plasmodium ovale, and Plasmodium malariae.

Dengue fever is an acute infectious disease caused by dengue virus, and is classified based on its clinical characteristics into classical dengue fever (CDF), which has a good prognosis, dengue hemorrhagic syndrome (DHF), which shows the tendency of hemorrhage, and dengue shock syndrome (DSS), which is the most severe form of the disease and is characterized by shock (Yoshihiro Hirabayashi, “Infectious disease syndrome I Ryoikibetsu Shokogun Shirizu No. 23”, 1999, pp. 145-149; Sabin, A. B., American Journal of Tropical Medicine and Hygiene, 1952, Vol. 2, pp. 30-50; Cohen, S. N. et al., Journal of Pediatrics, 1966, Vol. 68, pp. 448-456; Nimmannitya, S. et al., American Journal of Tropical Medicine and Hygiene, 1969, pp. 954-971).

Dengue virus, which is the pathogen of dengue fever, is about 40 to 60 nm in diameter and has an envelope. It has an about 11 kb positive single-stranded RNA, and belongs to the Flaviviridae family together with yellow fever virus, Japanese encephalitis virus and the like in terms of virology. In addition, it is known that dengue virus is classified into 4 serotypes (type 1 to type 4) based on the crossing-over of infection-neutralizing antibodies (Westaway, E. G. et al., Intervirology, 1985, Vol. 24, pp. 183-192; Chambers, T. J. et al., Annual Reviews Microbiology, 1990, Vol. 44, pp. 649-688).

The dengue virus vectors in nature are Aedine mosquitoes. Among them, Aedes aegypti mosquitoes which widely inhabit tropical areas become major carrying mosquitoes (Bancroft, T. L. Australasian Medical Gazette, 1906, Vol. 25, pp. 17-18).

Malaria is caused primarily by infection of one of four species of protozoa of the genus Plasmodium. The four species include: Plasmodium vivax, Plasmodium malariae, Plasmodium falciparum and Plasmodium ovale. Of these, Plasmodium falciparum produces the most pathogenic of the malarias and often results in death.

In malaria, the disease is such that infection followed by recovery does not confer meaningful protection to the individual despite a significant antibody response to several of the parasite proteins.

In the life cycle of the malaria parasite, a human becomes infected with malaria from the bite of a female Anopheles mosquito. The mosquito inserts its probe into a host and in so doing, injects a sporozoite form of Plasmodium falciparum, present in the saliva of the mosquito. The sporozoites which have been injected into the human host are cleared into a number of host tissue cells, including liver parenchyma cells (hepatocytes) and macrophages. This phase is known as the exoerythrocytic cycle because at this point in the life cycle the organism has not yet entered red blood cells. After entering hepatocytes, sporozoites undergo a transformation into trophozoites, which incubate and undergo schizogony, rupture and liberate tissue merozoites. This process takes approximately 7-10 days and, depending upon species, may repeat itself several times, during which time the host feels no effects. In Plasmodium falciparum, this repetition does not occur. After the incubation period, the liver or other tissue cells burst open (or bleb out) to release numerous merozoites into the bloodstream.

Shortly thereafter, certain of these blood borne merozoites invade red blood cells, where they enter the erythrocytic phase of the life cycle. Within the red blood cells, young plasmodia have a red nucleus and a ring-shaped, blue cytoplasm. The plasmodium divides into merozoites, which may break out of the red blood cell, enter other erythrocytes and repeat the multiplication process. This period lasts approximately 48 hours.

During this same 48 hour period of the erythrocytic cycle, male and female gametocytes are formed in the red blood cells. These gametocytes also burst out of the red blood cells along with the merozoites. It is during this period that the human host experiences the symptoms associated with malaria. The merozoites which burst forth from the red blood cells live for only a few hours in the bloodstream. The gametocytes live for several days or more in the host's bloodstream.

The gametocytes are capable of mating only in the mosquito. Thus, in order for Plasmdium falciparium to produce sporozoites for infecting a second human host, a mosquito must first bite a human host carrying gametocytes. These gametocytes mature into macrogametes, mate in the mosquito's stomach and produce a zygote. The zygote (ookinete) is active and moves through the stomach or the midgut wall. Under the lining of the gut, the ookinete becomes rounded and forms a cyst called an oocyst, in which hundreds of sporozoites develop. Sporozoites thereafter invade the entire mosquito and many of them enter the salivary glands where they are in a favorable position to infect the next host when the mosquito feeds on its blood. The life cycle thereafter simply repeats itself in another human host.

During the life cycle of Plasmdium falciparium, inhibition of invasion of the erythrocyte by the merozoite may be a key to developing an effective vaccine for malaria. Once the parasite has gained entry into the red cell, the parasite is no longer directly exposed to the immune system. In some embodiments it is desirable to include red blood cells in the co-culture of the invention, thereby allowing the observation of infectivity of or toxicity to RBCs.

In certain embodiments, gfp or dye-labeled sporozoites can be used as reporters of infections such as Malaria. Cell cultures containing such reporters can be used for (i) drug screening for liver-stage antimalarial agents, (ii) vaccine development and quality control, (iii) research, including identification of host factors for infection, and (iv) cultures of hibernating stages of certain types of malaria, for example plasmodium vivax.

HCV is an enveloped positive strand RNA virus in the Flaviviridae family. The single strand HCV RNA genome is approximately 9600 nucleotides in length and has a single open reading frame (ORF) encoding a single large polyprotein of about 3000 amino acids. In infected cells, this polyprotein is cleaved at multiple sites by cellular and viral proteases to produce the structural and non-structural (NS) proteins. In the case of HCV, the generation of mature nonstructural proteins (NS2, NS3, NS4A, NS4B, NS5A, and NS5B) is effected by two viral proteases. The first one, cleaves at the NS2/NS3 junction (henceforth referred to as NS2-3 protease); the second one is a serine protease contained within the N-terminal region of NS3 (NS3 protease) and mediates all the subsequent cleavages downstream of NS3, both in cis, at the NS3/NS4A cleavage site, and in trans, for the remaining NS4A/NS4B, NS4B/NS5A, NS5A/NS5B sites. The NS4A protein appears to serve multiple functions, acting as a cofactor for the NS3 protease and possibly assisting in the membrane localization of NS3 and other viral replicase components. The complex formation of the NS3 protease with NS4A seems necessary to the processing events, enhancing the proteolytic efficiency at all of the sites. The NS3 protein also exhibits nucleoside triphosphatase and RNA helicase activities. NS5B is a RNA-dependent RNA polymerase that is involved in the replication of HCV. For a review of HCV, see Houghton, “Chapter 32, Hepatitis C Viruses,” in: Fields Virology, 3rd ed., Fields et al. eds., pp 1035-1058, 1996, Lippincott-Raven Publishers, Philadelphia, Pa. ; Moradpour et al. Nat Rev Microbiol. 2007 5(6):453-63; and Knipe et al. Field's Virology. 5th Edition. 2007. ISBN-10:0781760607, ISBN-13:9780781760607 which are all incorporated herein by reference.

The HCV virus must infect liver cells in order to carry out its life cycle. After attachment of the viral envelope to receptors on cell membrane, the envelope fuses to the cellular membrane, thereby releasing the viral protein core into the target cell cytosol. After dissolution or breaking open of the protein coat, the viral RNA is released and employs the cell's ribosomes to create viral proteins. The NS proteins first direct synthesis of an antisense copy of the viral RNA, which serves as a template for the production of nascent plus sense viral RNA. Structural proteins and nascent plus sense viral RNA, in cooperation with certain NS proteins, assemble into progeny viral virus particles. These particles proceed membrane through the secretory pathway where they are eventually released out of the cell.

Examples 2-4 describe particular embodiments of the present invention where the technology described herein may be used to monitor HCV infectivity. Example 2 describes a fluorescence based reporter construct which may be employed to monitor HCV infection. Example 3 employs the HCV-dependent fluorescence relocalization (HDFR) cassette described in Example 2 to monitor the infection of hepatocytes. Example 4 employs the HDFR cassette and cellular co-culture methods to observe and quantify cell-to-cell infection by HCV. One of skill in the art will appreciate that the methods and constructs described in the Examples are only specific embodiments and that similar reporter constructs may be envisioned given the present disclosure (e.g., constructs with different fluorescent proteins, methods with different quantification methods, etc., other cells and cell cultures susceptible to infection by HCV, cells and cell cultures susceptible to infection by another virus or pathogen of interest as is appropriate)

Accordingly, the micropatterned co-cultures and reporter systems of the present invention may be employed to investigate various infectious diseases by, e.g., 1. screening therapeutics to identify drugs which prevent disease infection/replication; 2. identifying disease biomarkers (e.g., markers indicating disease infection, or host markers indicating susceptibility to infection or poor prognosis); 3. identification of target molecules on the host cell which may serve as receptors for disease infection (e.g., viral receptor); 4. validation of vaccines (e.g., determination of whether an attenuated viral vaccine is effective at preventing viral progression outside the liver); 5. determination of the sensitivity of parenchymal and/or non-parenchymal cells to certain therapeutics; 6. host specific sensitivity and regimin determination (e.g., producing a micropatterned co-culture with a specific patient's cells to determine the effective treatment regimin, dosage, or sensitivity to therapeutics.

### V. Methods of Treatment

Infectious Diseases and Disorders

Methods of treating infectious diseases or disorders are provided herein. “Treating” includes methods that cure, alleviate, relieve, alter, ameliorate, palliate, improve or affect the disorder, the symptoms of the disorder or the predisposition toward the disorder (e.g., a disorder or disease which is an infections disease such as from HCV, malaria, or other pathogens described herein). The methods can be used in vivo or on cells in culture, e.g., in vitro or ex vivo. For in vivo embodiments, the method is effected in a subject and includes administering the agent to the subject under conditions effective to attenuate or destroy virally infected cells or tissues of the subject.

Embodiments of the invention include methods of preparing and administering to a subject an agent (or agents) for preventing and/or treating viral infection in vivo. Preferred embodiments feature inhibitory compounds identified in any of the screening assay methodologies described herein.

Anti-viral agents in vitro can be further tested in vivo in animal models as well as in the micropatterned co-cultures of the invention. Data obtained from the cell culture and/or animal studies can be used in formulating an appropriate dosage of any given agent for use in humans. A therapeutically effective amount of an agent will be an amount that delays progression of or improves one or more symptoms of the condition, whether evident by improvement in an objective sign (e.g., viral titres) or subjective symptom of the disease. Certain factors may influence the dosage and timing required to effectively treat a subject (e.g., the severity of the disease or disorder, previous treatments, the general health and/or age of the subject, and other diseases present).

Anti-pathogen (anti-viral, anti-bacterial, anti-parasitic) agents identified according to the methodologies described herein can further be incorporated into pharmaceutical compositions and administered to subjects who have, or who are at risk of developing, infectious diseases or disorders. Such compositions will include one or more anti-viral agents and at least one pharmaceutically acceptable carrier (e.g., a solvent, dispersion medium, coating, buffer, absorption delaying agent, and the like, that are substantially non-toxic). Supplementary active compounds can also be incorporated into the compositions.

Pharmaceutical compositions are formulated to be compatible with their intended route of administration, whether oral or parenteral (e.g., intravenous, intradermal, subcutaneous, transmucosal (e.g., nasal sprays are formulated for inhalation), or transdermal (e.g., topical ointments, salves, gels, patches or creams as generally known in the art). The compositions can include a sterile diluent (e.g., sterile water or saline), a fixed oil, polyethylene glycol, glycerine, propylene glycol or other synthetic solvents; antibacterial or antifungal agents such as benzyl alcohol or methyl parabens, chlorobutanol, phenol, ascorbic acid, thimerosal, and the like; antioxidants such as ascorbic acid or sodium bisulfite; chelating agents such as ethylenediaminetetraacetic acid; buffers such as acetates, citrates or phosphates; and isotonic agents such as sugars (e.g., dextrose), polyalcohols (e.g., manitol or sorbitol), or salts (e.g., sodium chloride). Liposomal suspensions can also be used as pharmaceutically acceptable carriers. Preparations of the compositions can be formulated and enclosed in ampules, disposable syringes or multiple dose vials. Where required (as in, for example, injectable formulations), proper fluidity can be maintained by, for example, the use of a coating such as lecithin, or a surfactant. Absorption of the active ingredient can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Where oral administration is intended, the agent can be included in pills, capsules, troches and the like and can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring

Anti-viral agents of the invention can be formulated for oral or parenteral administration in dosage unit form (i.e., physically discrete units containing a predetermined quantity of active compound for ease of administration and uniformity of dosage). Toxicity and therapeutic efficacy of compounds can be determined by standard pharmaceutical procedures in the co-cultures and/or systems of the invention or experimental animals. One can, for example, determine the LD₅₀ (the dose lethal to 50% of the population) and the ED₅₀ (the dose therapeutically effective in 50% of the population), the therapeutic index being the ratio of LD₅₀:ED₅₀. Agents that exhibit high therapeutic indices are preferred. Where an agent exhibits an undesirable side effect, care should be taken to target that agent to the site of the affected tissue (the aim being to minimize potential damage to unaffected cells and, thereby, reduce side effects). Toxicity and therapeutic efficacy can be determined by other standard pharmaceutical procedures.

Data obtained from the cultures and/or systems of the invention (optionally, in combination with animal studies) can be used in formulating an appropriate dosage of any given agent for use in humans. A therapeutically effective amount of an agent will be an amount that delays progression of or improves one or more symptoms of the infectious disease or disorder, whether evident by improvement in an objective sign (e.g., viral titres) or subjective symptom of the disease. Certain factors may influence the dosage and timing required to effectively treat a subject (e.g., the severity of the disease or disorder, previous treatments, the general health and/or age of the subject, and other diseases present).

Agents identified and administered according to the methods described here can be small molecules (e.g., peptides, peptidomimetics (e.g., peptoids), amino acid residues (or analogs thereof), polynucleotides (or analogs thereof), nucleotides (or analogs thereof), or organic or inorganic compounds (e.g., heteroorganic or organometallic compounds). Typically, such molecules will have a molecular weight less than about 10,000 grams per mole (e.g., less than about 7,500, 5,000, 2,500, 1,000, or 500 grams per mole). Salts, esters, and other pharmaceutically acceptable forms of any of these compounds can be assayed and, if a desirable activity is detected, administered according to the therapeutic methods described herein. Exemplary doses include milligram or microgram amounts of the small molecule per kilogram of subject or sample weight (e.g., about 1 μg-500 mg/kg; about 100 μg-500 mg/kg; about 100 μg-50 mg/kg; 10 μg-5 mg/kg; 10 μg-0.5 mg/kg; or 1 μg-50 μg/kg). While these doses cover a broad range, one of ordinary skill in the art will understand that therapeutic agents, including small molecules, vary in their potency, and effective amounts can be determined by methods known in the art. Typically, relatively low doses are administered at first, and the attending physician or veterinarian (in the case of therapeutic application) or a researcher (when still working at the clinical development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The pharmaceutical compositions can be included in a container, pack, or dispenser together with instructions for administration.

### VI. Kits

The invention further provides kits for performing the screening or monitoring methods described above. Typically, such kits contain a culture of the invention and also typically contain labeling providing directions for use of the kit. Cultures can be infected or uninfected. Infectious agent can be separately packaged in the kit. Cultures are preferably preserved, e.g., packages in call preservation media or cryopreserved. The term labeling refers to any written or recorded material that is attached to, or otherwise accompanies a kit at any time during its manufacture, transport, sale or use. For example, the term labeling encompasses advertising leaflets and brochures, packaging materials, instructions, audio or videocassettes, computer discs, as well as writing imprinted directly on kits. Kits can optionally contain one or more suitable control.

In one aspect, the invention comprises screening assay kits for identifying, or validating anti-infective therapeutics, e.g., small molecule therapeutics (e.g., anti-parasitic agents, anti-viral agents and the like), vaccines, etc. In another aspect, the invention provides diagnostic kits, for example, research, detection and/or diagnostic kits (e.g., for performing biomarker detection, etc.). Preferably, the kit is labeled with instructions for performing the intended application, for example, for performing screening assays, biomarker detection, etc. Exemplary screening assays are described herein. Exemplary biomarker are those identified according to the methods described herein.

Other exemplary kits of the invention contain one or more reporter system components, for example, a reporter vector, optionally packaged and/or labeled with instructions for use.

An advantage of the present invention is that micropatterned co-cultures with primary hepatocytes are able to take up the HCVpp and HCVcc. A further advantage of the present invention is that the co-cultures are able to produce de novo particles in vitro for several weeks. Yet another advantage of the present invention is that an evaluation of the afore-mentioned steps can be done by non-destructive reporter systems. Another advantage of the present invention is that the co-culture-reporter combination can be used in several applications such as, for example, determining the efficacy of small molecules for drug discovery. Yet another advantage of the present invention is that the cell cultures can be used with actual patient isolates/clinical isolates with applications in detecting and/or real time monitoring of in vivo infection efficiency.

While the micropatterned co-culture systems described herein are particularly suited for drug discovery and vaccine discovery efforts, it will be appreciated that the basic methodologies may be applied to other general models of pathogenesis, for example, other tissue models or engineered human tissues (e.g., ES cell-derived differentiated cell culture models) coupled with the infectious disease model technology described herein. The above-described systems are likewise amenable to development of species-specific test systems (e.g., systems to study non-human animal pathogenesis, porcine pathogenesis, equine pathogenesis, feline pathogenesis, bovine pathogenesis, canine pathogenesis, and the like). The above-described systems (e.g., micropatterned cultures and/or systems, etc) are also amenable to implantation into laboratory animals, for example mice, to generate animal in vivo models of human pathogenesis (e.g., “humanized” animal responses). For example, infected three-dimensional micropartterened cultures can me implanted in a model animal (e.g. a mouse) to study further in vivo aspects of pathogenesis.

The foregoing disclosure teaches to those of skill in the art the aspects of the invention including how to make and use the invention. The present invention is further illustrated in the following examples, which should not be construed as limiting.

### Examples

### Example 1

### Disease Platform for HCV Drug Development

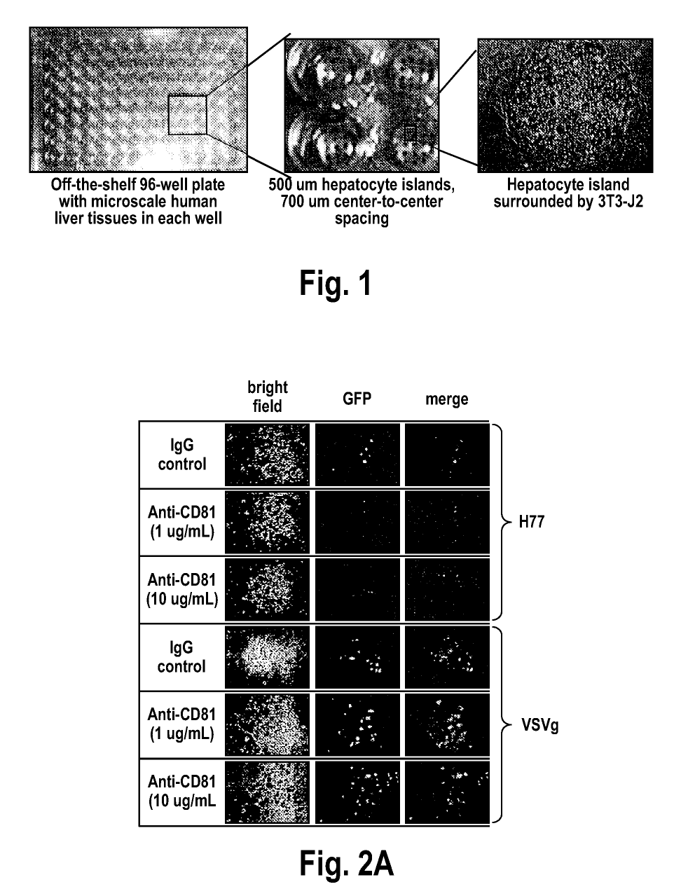

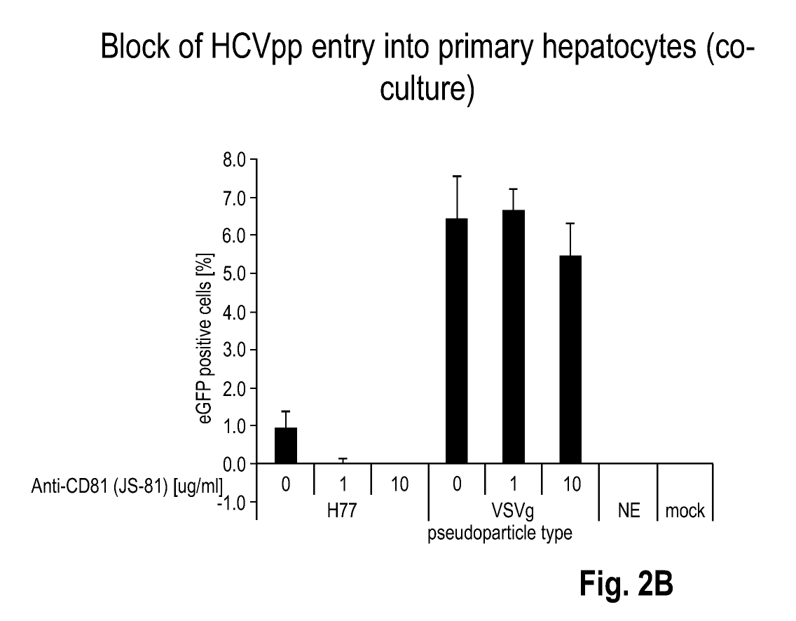

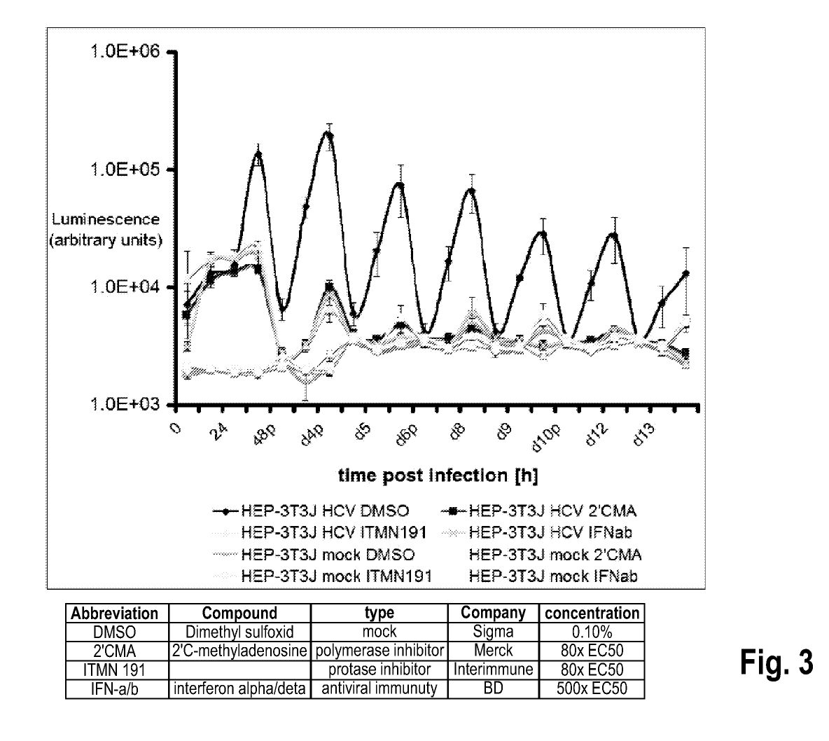

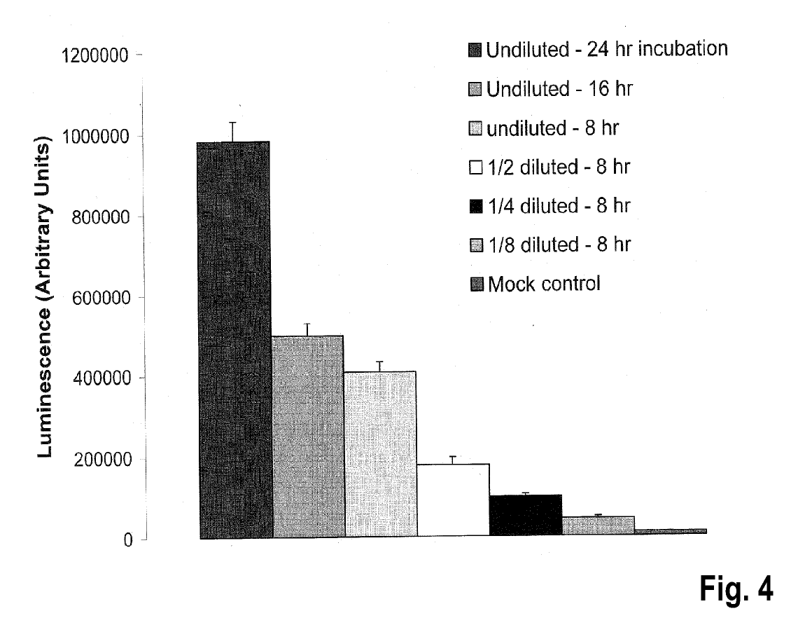

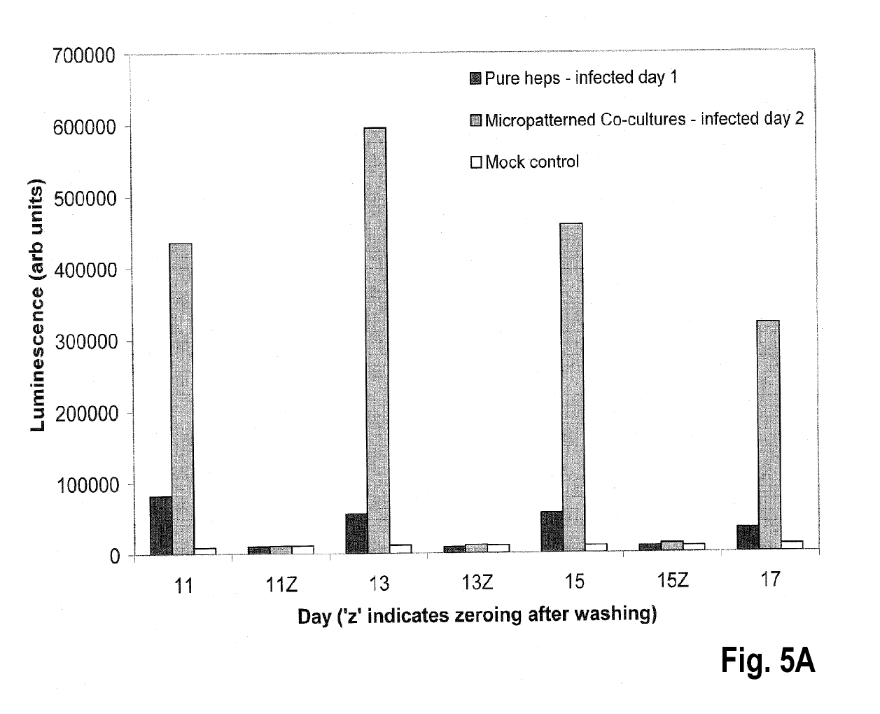

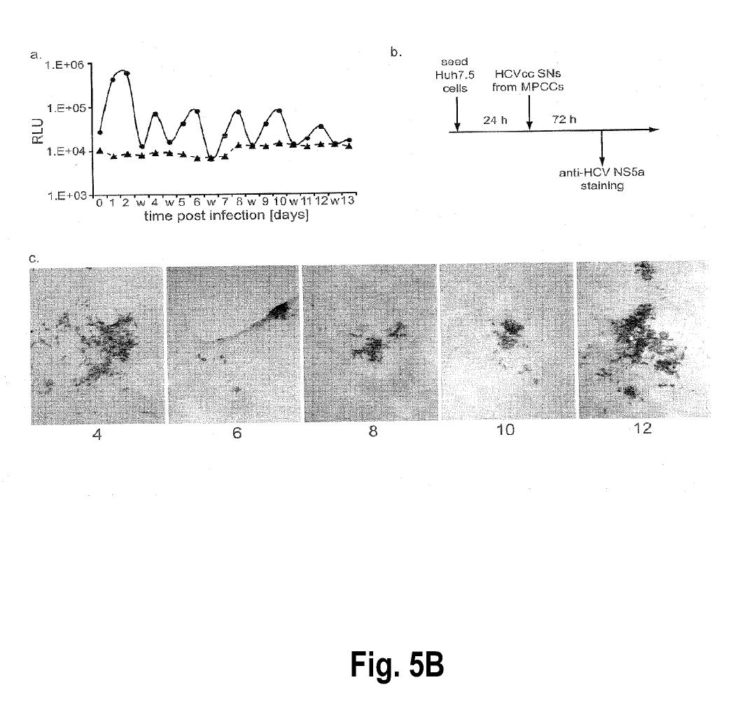

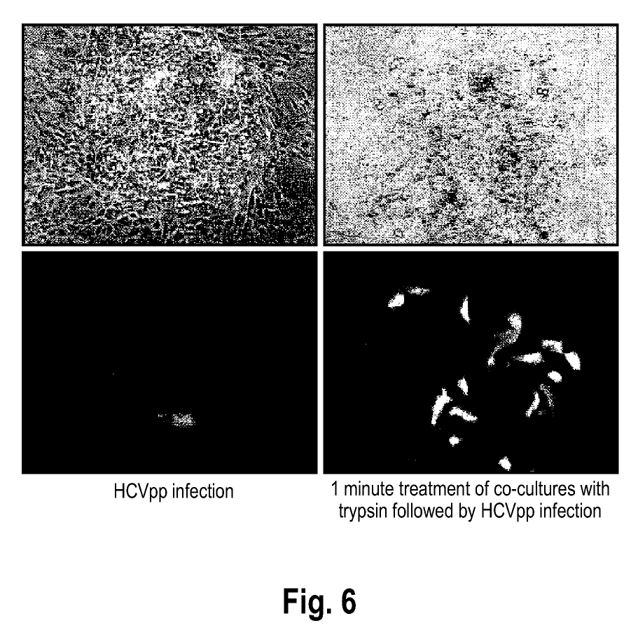

This Example presents the development and optimization of a disease platform for HCV drug development that couples micropatterned co-cultures of primary human hepatocytes and stromal cells in a multi-well format (FIG. 1) with highly sensitive viral reporter systems and optimized treatment protocols. The developed human liver platform can: a) Support entry of HCV-like particles via receptor-mediated processes (FIG. 2), and b) Produce and release infectious HCV particles into the culture supernatant for several weeks in vitro, and that such release can be blocked by treatment of cultures with small molecule inhibitors of viral proteins (FIG. 3). It was also found that infection of micropatterned co-cultures is dependent on the dose and time of exposure to the initial inoculum (FIG. 4), and magnitude of HCV infection in micropatterned co-cultures is 4-6 fold higher than in pure hepatocyte monolayers, the current gold standard in the field (FIG. 5a). Lastly, it was discovered that pre-treatment of micropatterned co-cultures with trypsin/EDTA for 1 minute significantly enhanced (16-20 fold) subsequent entry of HCV-like particles over trypsin-free controls (FIG. 6). This result was unexpected since the trypsin/EDTA treated co-cultures show a viral entry which is enhanced beyond that of fresh human hepatocytes (generally considered a gold standard).

The human liver platform (96-well standard tissue cultures plates amenable to robotic screening) containing micropatterned co-cultures along with the novel trypsin/EDTA treatment protocol (to significantly enhance HCV entry into cells) can be applied to other pathogens that infect the liver (i.e. malaria, HBV, etc.). Furthermore, it is anticipated that in the future, micropatterned co-cultures created from hepatocytes derived from induced pluripotent stem cells (iPS) from patient biopsies, can be infected with HCV or other pathogens to provide patient-specific testing of drugs.

### Example 2

### A Fluorescent-Based Reporter for HCV Infection

A novel cell-based fluorescent reporter system has been developed to detect infection of unmodified HCV genomes. The example system described here uses an HCV-dependent fluorescence relocalization (HDFR) cassette that comprises a fluorescent protein (e.g. EGFP, mCherry or TagRFP), an SV40 nuclear localization sequence (NLS), and a C-terminal mitochondrial-targeting domain (IPS) derived from the interferon-beta promoter stimulator 1 protein, IPS-1. IPS-1 is a known cellular substrate for the HCV NS3-4A protease, and IPS-1 mutation C508Y has been shown to abolish cleavage. In HCV-infected cells the HDFR cassette is processed by the viral NS3-4A protease, resulting in translocation of the fluorescent protein from the mitochondria to the nucleus. Using a lentivirus based expression system to stably express the HDFR cassette, we have successfully established this system in the highly HCV permissive human hepatoma cell line, Huh-7.5 and in adult primary human hepatocytes maintained in culture as micropatterned co-cultures (MPCC). This reporter system can be used to identify individual infected cells significantly earlier than established antibody-based methods, it can detect infection without sample processing or cell fixation/lysis, and it can easily visualize live cell infection in real time for study in conjunction with other biological processes.

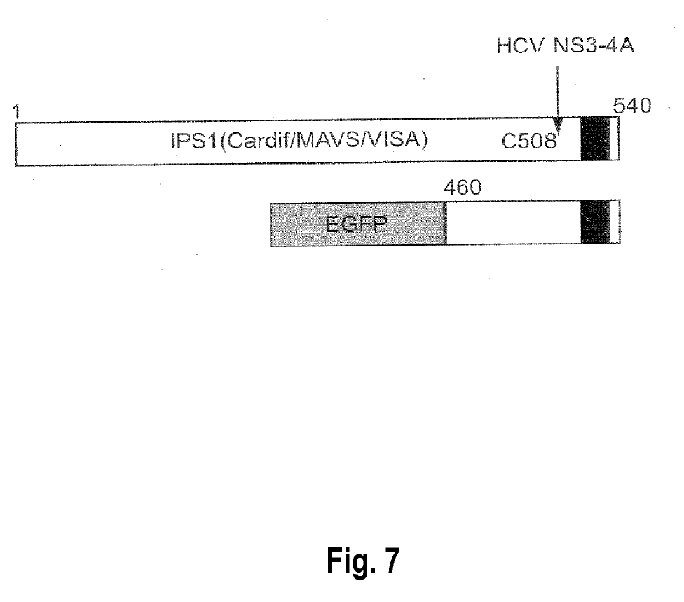

FIG. 7 depicts the development of HCV-dependent Fluorescence Relocalization (HDFR) reporter cells. IPS1 (also know as Cardif/MAVS/VISA) is a mitochondrial protein cleaved by HCV NS3/4A protease in the HCV infection pathway. Mitochondrially anchored IPS1/Cardif is cleaved and activated by HCV NS3/4A protease following sensing of viral dsRNA by RLH. Activated IPS1/Cardif, in turn, recruits appropriate IKKs to activate NF-kappaB and IRF, resulting in the induction of type I IFN in infected cells. See e.g., Meylan et al. Nature (2006) 442, 39-44. EGFP-IPS is a fragment of IPS/Cardif comprising the NS3/4A cleavage site, fused to EGFP.

Fluorescence microscopy was used to demonstrate the effectiveness of the HDFR reporter system. In one experiment, Huh7.5 cells stably expressing the EGFP-IPS construct were infected with HCV and examined for the pattern of immunofluorescence staining. Cleavage by HCV NS3/4A protease results in localization of the EGFP-IPS construct to the mitochondria. To demonstrate mitochondrial localization, cells were immunostained with an antibody against COXIV, a protein that localizes to the mitochondria. Green fluorescence showed subcellular localization of EGFP-IPS; red fluorescence showed localization of COXIV; and blue fluorescence showed nuclei. An overlay of the green and red fluorescence demonstrates that EGFP-IPS colocalizes with COXIV and thus with the mitochondria.

In another experiment, immunofluorescence staining against HCV NS5A protein (a marker of HCVcc infection) was examined in HCVcc infected Huh7.5 cells stably expressing EGFP-IPS. Localization (in green) of the EGFP-IPS construct relative to the cell nucleus (blue) was first detected followed by detection of the location of NS5A (red). In HCV NS5A positive cells, EGFP-IPS relocalizes from the mitochondria to the cytoplasm. In HCV NS5A negative cells, EGFP-IPS remains localized to the mitochondria.

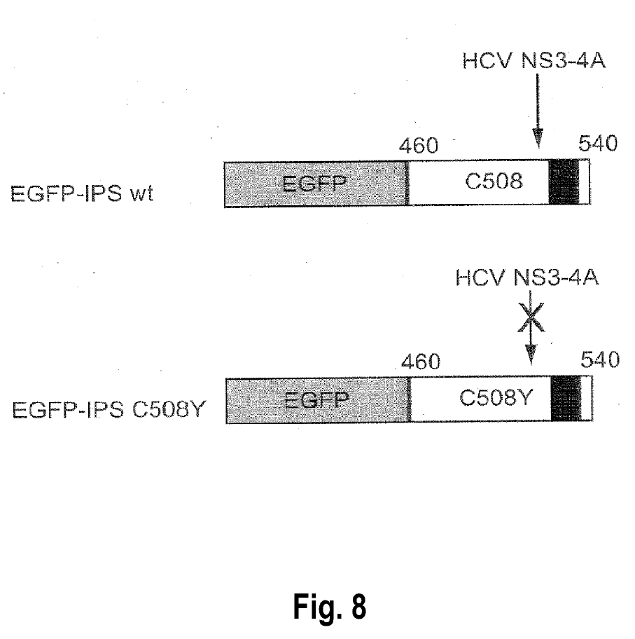

Finally, it was demonstrated that relocalization of EGFP-IPS depends upon the NS3-4A cleavage site. In this experiment, immunofluorescence staining against HCV NS5A protein was examined in HCVcc infected Huh7.5 cells stably expressing EGFP-IPS C508Y (cleavage site mutation compared to normal schematically in FIG. 8). Cellular localization of EGFP-IPS C508Y (in green) relative to the NS5A viral protein (red) showed EGFP-IPS C508Y is localized to the mitochondria. The results indicate that the C508Y mutation disrupts the NS3-4A cleavage site, and thus prevents relocalization of EGFP-IPS C508Y to the cytoplasm in HCVcc infected cells.

### Example 3

### Live Cell Imaging of HCV Infection

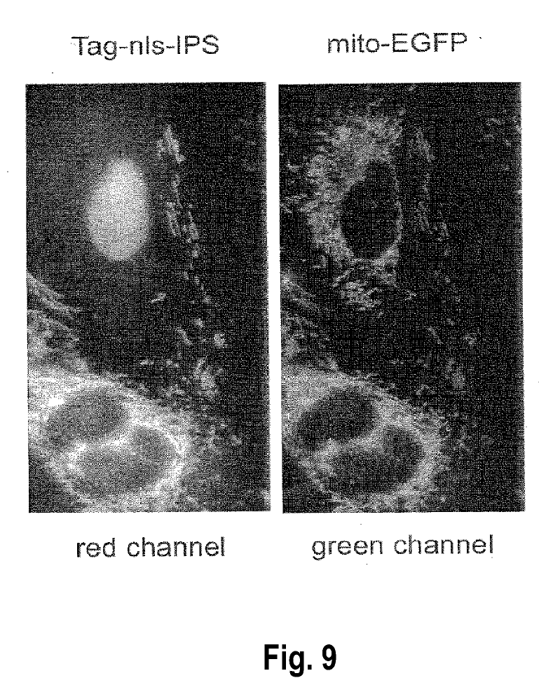

Live cell imaging of HCV infection was demonstrated using the Huh7.5 cell line stably expressing a modified red fluorescent version of EGFP-IPS (Tag-RFP-nls-IPS) and mito-EGFP. TagRFP-nls-IPS encodes TagRFP (a bright red fluorescent protein), a SV40 nuclear localization sequence (nls), and the mitochondrial targeting sequence from the C-terminus of IPS-1. Mito-EGFP encodes the mitochondrial targeting sequence from subunit VIII of cytochrome-c oxidase. Localization of Mito-EGFP to the mitochondria, shown as green staining surrounding cell nuclei. Following HCVcc infection of the resultant cell line, relocalization of the TagRFP-nls-IPS from the mitochondria to the nucleus was visualized (red/violet staining of cell nuclei) as a result of cleavage by the HCV NS3/4A protease and subsequent transport to the nucleus via the nls. Mito-EGFP localization is unaltered by HCV infection and serves as a marker for the mitochondria. The stark visualization of the red/violet nucleus shows clearly the relocalization of TagRFP-nls-IPS upon HCV infection. FIG. 9 depicts live cell imaging of infected cells in greyscale, with the TagRFP-nls-IPS staining mitochondria (light grey), Mito-EGFP staining mitochondria (dark grey), and TagRFP-nls-IPS localized to nucleus upon HCV infection (darkest grey).

### Example 4

### Use of HDFR to Detect Infection with HCVcc and Sera/Plasma from HCV+ Patients in MPCC

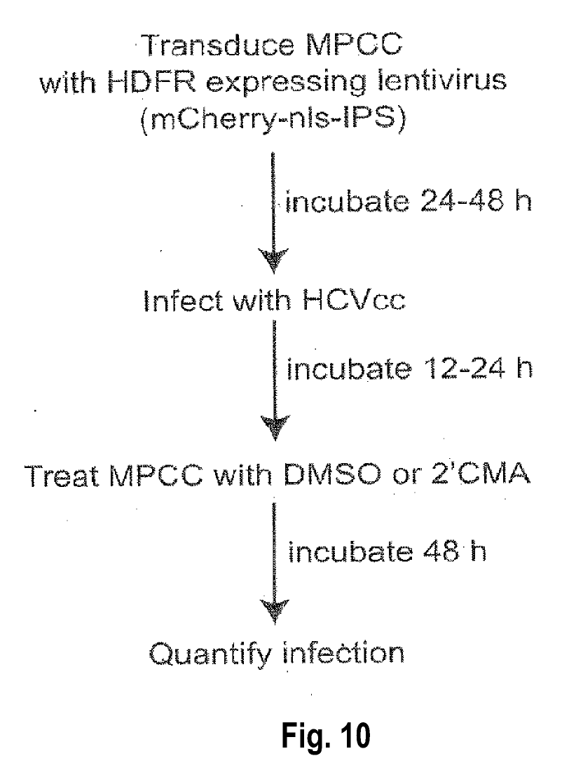

The cell-based fluorescent reporter system has recently proved particularly useful in studying HCV infection of primary cells. Traditionally, HCV-specific antigens have been extremely difficult to detect in infected primary cell cultures. This may be due to low permissiveness of these cells to infection, low intrinsic replication rate of this persistent virus, or high intrinsic liver cell fluorescence, preventing detection of weak signals. In contrast, the reporter system described here allows rapid detection and quantification of primary cell infection on a single cell basis. MPCC were transduced with a lentivirus encoding an mCherry-based HDFR cassette and subsequently infected with HCV cell culture virus (HCVcc). A flow diagram exemplifying this procedure is depicted in FIG. 10. Redistribution of mCherry fluorescence to the nucleus was detected in DMSO-treated MPCCs, but not in the presence of the HCV polymerase inhibitor (2′CMA) or the IPS-1 mutation (C508Y), indicating that the reporter relocalization is HCV dependent.

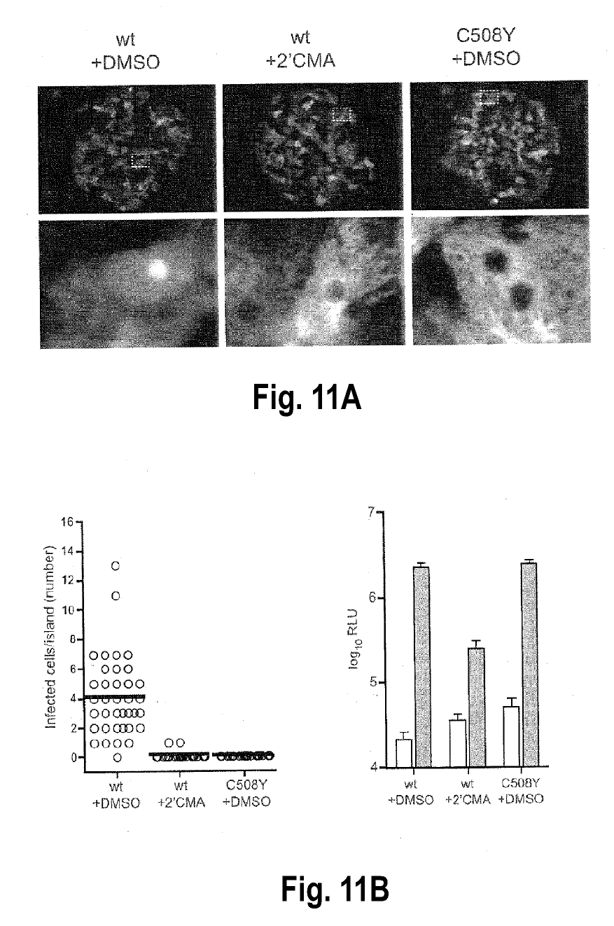

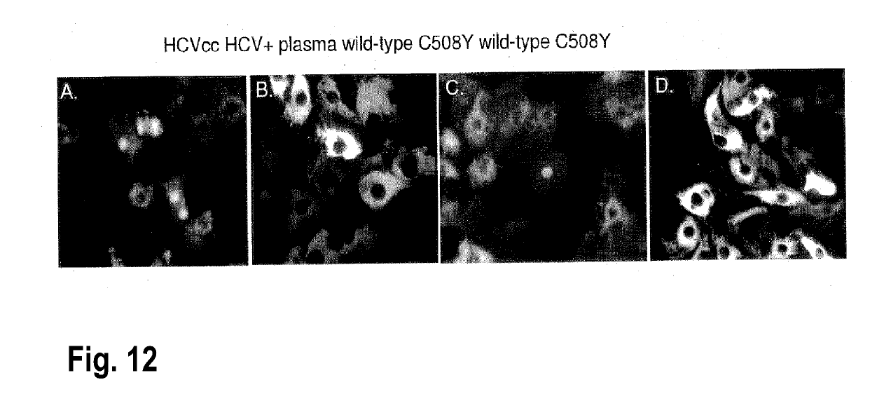

FIG. 11A (top images) depicts low magnification (10×) fluorescent images of adult primary hepatocytes in MPCCs transduced with lentivirus expressing the mCherry-nls-IPS HDFR cassette and subsequently infected with HCVcc in the presence of DMSO or the HCV polymerase inhibitor 2′CMA. Higher magnification (40×, FIG. 11A, bottom images) clearly shows the nuclear localization of mCherry-nls-IPS FIG. 11B further shows successful results measuring infected cells and RLU (relative luminescence units) in DMSO-treated MPCCs, cultures treated with HCV polymerase inhibitor (2′CMA), and cells having the IPS-1 mutation (C508Y). The left graph shows quantification of nuclear mCherry fluorescence of HCVcc infected MPCC islands plotted as the number of positive cells per island in the presence of DMSO or 2′CMA or in DMSO treated MPCC islands transduced with a mutant version of mCherry-nls-IPS (C508Y). The right graph shows Gaussia luciferase activity detected in the cell culture supernatants at 48 h post infection of MPCC infected with HCVcc secreted gaussia reporter virus. FIG. 12 shows that primary hepatocytes in MPCCs can be infected, albeit are very low frequency (<1/30000 cells) with sera/plasma from HCV positive patients.

### Example 5

### Use of HDFR to Study Cell-to-Cell Transmission of HCVcc

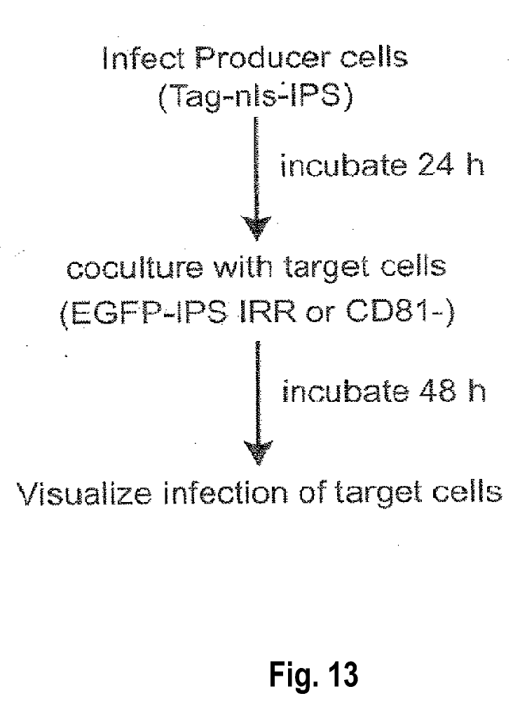

It is now well established that several essential receptor and co-receptor molecules, including CD81, SR-B1, CLDN1, and most recently OCLN, mediate HCV entry into naïve cells. Recently, an additional cell contact-dependent entry mechanism has been described, which does not require CD81 and occurs in the absence of the production of cell-free virus. This cell-to-cell transmission route may play an important role in viral persistence. A visual assay is established herein to study the cell-to-cell route of virus infection using the reporter cell line technology described herein. The assay employs two distinct populations of reporter cells designated the “producer” and the “target”. The producer and target cells are distinguished by the expression of HDFR cassettes encoding spectrally distinct fluorescent proteins (e.g., EGFP and TagRFP). Furthermore, the producer cell line is permissive to both cell-free and cell-to-cell routes of virus infection, while the target is permissive to only cell-to-cell routes of infection due to silencing of one (or more) essential HCV entry molecules. The producer cells are pre-infected with HCVcc under conditions ensuring efficient infection, and subsequently mixed with target cells under conditions permitting cell-to-cell virus infection of target cells. Cell-to-cell infection of target cells is then assessed as described above in live or fixed cells using fluorescence microscopy or flow cytometry-based methods. FIG. 12 is a flow chart depicting an exemplary cell-cell infection assay. FIGS. 13 through 18 further depict the results of experiments using the cell infection assay, demonstrating that the described system may be used effectively to monitor HCV infection and, in particular, cell-to-cell infection.

In a first experiment, HCVcc infection of an Huh7.5 cell line stably expressing TagRFP-nls-IPS caused relocalization of TagRFP-nls-IPS from the mitochondria to the nucleus as evidenced by significant red/violet staining of nuclei 48 hours post infection (versus staining surrounding nuclei in uninfected cells).

In a second experiment, HCVcc infection of EGFP-IPS derived Huh7.5 cell lines in which the expression of the HCV entry receptor CD81 has been knocked down using either an IRR shRNA (IRR cells) or one specifically targeting CD81 (CD81− cells) was monitored. Knockdown of CD81 expression renders cells non-permissive to HCVcc infection as indicated by an absence of EGFP-IPS relocalization (48 hours post infection).

In a third experiment, uninfected TagRFP-nls-IPS and EGFP-IPS (IRR) Huh7.5 cell lines are co-cultured. In the absence of HCVcc infection, relocalization is not observed in either of the cell lines. In the presence of HCVcc infection, nearly complete relocalization of EGFP-IPS is observed.

In a final experiment, uninfected TagRFP nls-IPS and EGFP-IPS (CD81−) Huh7.5 cell lines are co-cultured. In the absence of HCVcc infection, relocalization in not observed in either of the cell lines. Following HCVcc infection, although significantly fewer EGFP-IPS (CD81−) cells show relocalization of EGFP-IPS compared to EGFP-IPS (IRR) cells (compared with above data), infection is readily observed, despite the non-permissiveness of these cells to cell-free virus infection (see “second experiment”, described above).

### EQUIVALENTS

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the invention, and exclusive use of all modifications that come within the scope of the appended claims is reserved. It is intended that the present invention be limited only to the extent required by the appended claims and the applicable rules of law.

All literature and similar material cited in this application, including, patents, patent applications, articles, books, treatises, dissertations, web pages, figures and/or appendices, regardless of the format of such literature and similar materials, are expressly incorporated by reference in their entirety. In the event that one or more of the incorporated literature and similar materials differs from or contradicts this application, including defined terms, term usage, described techniques, or the like, this application controls. Such equivalents are intended to be encompassed by the following claims.

1-40. (canceled)

41. A method of identifying a candidate anti-infective agent for use in treating liver pathogens, the method comprising
(a) obtaining a micropatterned co-culture (MPCC) comprising a population of primary hepatocytes and one or more populations of non-parenchymal cells, wherein the hepatocytes and non-parenchymal cells are micropatterned such that hepatic phenotype and metabolic function are supported;
(b) exposing the MPCC of (a) to a test agent and a liver pathogen;
(c) culturing the MPCC for a time sufficient to permit infection of the hepatocytes with the liver pathogen; and
(d) detecting within the hepatocytes an indicator of infection,
wherein a decrease in the indicator of infection as compared to a suitable control indicates that the test compound is a candidate anti-infective agent.

42. The method of claim 41, wherein step (b) comprises exposure to liver pathogen before exposure to test agent.

43. The method of claim 41, wherein step (b) comprises exposure to test agent before exposure to liver pathogen.

44. A method of validating a candidate vaccine for use in preventing disease caused by liver pathogens, the method comprising
(a) obtaining a micropatterned co-culture (MPCC) comprising a population of primary hepatocytes and one or more populations of non-parenchymal cells, wherein the hepatocytes and non-parenchymal cells are micropatterned such that hepatic phenotype and metabolic function are supported;
(b) exposing the MPCC of (a) to a vaccine of attenuated pathogen and a liver pathogen;
(c) culturing the MPCC for a time sufficient to permit infection of the hepatocytes with the liver pathogen; and
(d) detecting within the hepatocytes an indicator of infection,
wherein a decrease in the indicator of infection as compared to a suitable control indicates that the test compound is a candidate anti-infective agent.

45. The method of claim 44, wherein the MPCC further includes immune cells, and wherein step (c) comprises culturing the MPCC for a time sufficient to permit infection of the hepatocytes with the liver pathogen and/or immune response of the RBCs to the liver pathogen; and wherein step (d) comprises detecting the indicator of infection and/or, measuring the immune response to the attenuated pathogen.

46. The method of claim 44, wherein step (b) comprises exposure to liver pathogen before exposure to vaccine.

47. The method of claim 44, wherein step (b) comprises exposure to vaccine before exposure to liver pathogen.

48. The method of claim 41, wherein the liver pathogen is a malaria parasite.

49. The method of claim 48, wherein the liver pathogen is selected from the group consisting of Plasmodium falciparum, Plasmodium vivax, Plasmodium ovule and Plasmodium malariae.

50. (canceled)

51. The method of claim 41, wherein the indicator is an indicator of pathogen infection, replication or toxicity in hepatocytes or erythrocytes.

52. (canceled)

53. The method of claim 41, wherein the indicator is an immunofluorescent pathogen or immunofluorescent pathogen protein.

54. (canceled)

55. The method of claim 41, wherein the indicator is detected using an automated image analysis.

56. The method of claim 41, wherein the test agent is selected from the group consisting of a peptide, a polypeptide, an antibody, an adnectin, a peptidomimetic, a small molecule, an oligonucleotide and a polynucleotide.

57. The method of claim 41, further comprising testing the toxicology of the test agent.

58. The method of claim 41, wherein at least 0.01% of the pathogens infect hepatocytes.

59. (canceled)

60. The method of claim 41, wherein the control is a comparable uninfected population or infection-inhibited population of hepatocytes.

61. (canceled)

62. The method of claim 41, wherein at least one of the non-parenchymal cell populations comprises stromal cells or hepatic non-parenchymal cells.

63. The method of claim 62, wherein the stromal cells are fibroblasts or fibroblast-derived cells.

64. (canceled)

65. The method of claim 41, wherein the culture further comprises a population of immune cells.

66. (canceled)

67. The method of claim 62, wherein the hepatic non-parenchymal cells are selected from the group consisting of Kupffer cells, Ito cells, endothelial cells, stellate cells, cholangiocytes (bile duct cells) and hepatic natural killer (NK) cells (pit cells).

68. (canceled)

69. The method of claim 41, wherein the hepatocytes are micropatterned on a plurality of microspot islands or microwells comprising a cell adhesion substrate.

70. The method of claim 41, wherein the non-parenchymal cell population borders the population of micropatterened hepatocytes.

71. The method of claim 41, wherein the cells are cultured on a biopolymer scaffold.

72. (canceled)

73. The method of claim 41, wherein the micropatterened hepatocyte population is a 3-dimensional micropattern.

74. The method of claim 69, wherein the cell adhesion substrate comprises a plurality of substrates.

75. The method of claim 69, wherein the substrate is selected from the group consisting of collagen, fibronectin, vitronectin, laminin, Arg-Gly-Asp (RGD) peptide, Tyr-Ile-Gly-Ser-Arg (YIGSR) peptide, gycosaminoglycans (GAGs), hyaluronic acid (HA), integrins, ICAMs, selectins, cadherins and cell surface protein-specific antibodies.

76. The method of claim 41, wherein the non-parenchymal cells are patterned.

77. The method of claim wherein the non-parenchymal cells are cultured on an inhibitory substrate.

78. The method of claim 77, wherein the inhibitory substrate is selected from the group consisting of bovine serum albumin, poly hydroxyethyl-mathacrylate, polyacrylamide, polymethylacrylate, triblock polymer, pluronics and surfactants.

79. The method of claim 41, wherein the MPCC is housed in a bioreactor.

80. The method of claim 79, wherein the bioreactor controls gas exchange across the cell populations.

81. The method of claim 80, wherein the bioreactor controls an oxygen gradient across the cell populations.
